(19) United States
(12) Patent Application Publication
(10) Pub. No.: US 2015/0219618 A1
(43) Pub. Date: Aug. 6, 2015

(54) MANIPULATION OF MICROPARTICLES IN LOW FIELD DIELECTROPHORETIC REGIONS

(71) Applicant: BIOLOGICAL DYNAMICS, INC., SAN DIEGO, CA (US)

(72) Inventors: Rajaram Krishnan, San Diego, CA (US); David Charlot, San Diego, CA (US); Eugene Tu, San Diego, CA (US); Jerry Lu, San Diego, CA (US)

(21) Appl. No.: 14/415,546

(22) PCT Filed: Jul. 18, 2013
(86) PCT No.: PCT/US13/51158

§ 371 Date: Jan. 16, 2015

Related U.S. Application Data
(60) Provisional application No. 61/672,949, filed on Jul. 18, 2012.

(51) Int. Cl. G01N 33/483; C12Q 1/68; G01N 27/447; G01N 33/569

(52) U.S. Cl. CPC G01N 33/4836; G01N 33/56911; C12Q 1/6837; G01N 27/44721

(57) ABSTRACT

The present invention includes methods, devices and systems for isolating a target biological material from a biological sample. In various aspects, the methods, devices and systems may allow for a rapid procedure that requires a minimal amount of material and/or results in isolated target biological material from complex fluids such as blood or environmental samples.

### CROSS-REFERENCE

This application claims the benefit of U.S. Application No. 61/672,949, filed Jul. 18, 2012, which is incorporated by reference in its entirety.

### BACKGROUND OF THE INVENTION

Biomarker identification efforts have expanded greatly in recent years. In addition to gaining novel techniques of diagnosing diseases or identifying disease states, expanded biomarker identification has the potential of adding new tools for monitoring disease progression and treatment efficacy. However, microarray-based methods, including proteomics, gene-based microarrays, imaging techniques and next-generation sequencing, have all generated massive amounts of data that have not translated into clinical practice. Validation of potential biomarkers is especially lacking, where techniques for rapid and efficient clinical assays have not kept pace. In addition, improved sample preparation methods and methods for isolating target markers or other biological material are also lacking. Especially acute where minute sample volumes or amounts are available, the inability to efficiently isolate sample material can hamper downstream biomarker identification efforts.

### SUMMARY OF THE INVENTION

In some instances, the present invention fulfills a need for improved methods of biological material isolation from biological samples. Particular attributes of certain aspects provided herein include a total sample preparation time of less than about one hour, with hands-on time of less than about one minute. In some embodiments, the present invention can be used to isolate biological material from dilute and/or complex fluids such as blood or environmental samples. In other aspects, the present invention can use small amounts of starting material, achieve the purification of target biological materials, and is amenable to multiplexed and high-throughput operation.

In some aspects, the target biological material may comprise cellular components, including but not limited to organelles, mitochondria, apoptotic bodies, endoplasmic reticulum, cell surface membranes, golgi bodies, nuclei or other nuclear structures with attached nucleic acids (e.g. nucleolus, chromosomes, chromatin or other subnuclear bodies), the nuclear envelope and/or other cell-associated structures.

In other aspects, the target biological material may comprise large exosomes and other non-cell or extracellular bodies, including but not limited to micelles, large chylomicrons, blood clots, plaques, protein aggregates (e.g. beta-amyloid plaques or tau protein) that are larger than about 800 nm or about 1 micron in diameter or size. In some aspects, the large exosomes captured using the methods described herein may be used for further mRNA, miRNA, nucleic acid, protein, peptide and/or enzyme analysis.

In another aspect, the target biological material may comprise bacteria, protists, nematodes, parasites or other microbiological entities. In some aspects the microbiological target material may be further processed to obtain additional target biological material, including microbiological cell material including but not limited to, e.g. bacterial membranes, ribosomes and other bacterial- or microbiological-associated structures.

In yet another aspect, the isolated target biological material may be used to detect, identify or monitor a variety of disease states, including cardiovascular diseases, cancer, metabolic diseases and/or prion-based diseases. In yet other aspects, the isolated target biological material may also be used to monitor other physiological states, including catabolic states, drug delivery efficacy and mechanisms and/or the liquid monitoring of tissue damage.

In one aspect, described herein is a method for isolating a target biological material from a biological sample, the method comprising: (a) applying the biological sample to a device, the device comprising an array of electrodes; (b) creating dielectrophoretic (DEP) low-field and dielectrophoretic (DEP) high-field regions on the array; and; (c) selectively retaining the target biological material on the DEP low-field region. In some embodiments, dielectrophoretic (DEP) low-field, DEP intermediate-field and DEP high-field regions are created on the array. In some embodiments, the target biological material is at least 800 nm, at least 900 nm, at least 1000 nm, at least 1100, at least 1200, at least 1300, at least 1400, at least 1500 nm, at least 2000 nm, at least 2500 nm, at least 3000, about 800-10000 nm, about 800-5000 nm, about 800-4000 nm, about 800-3000 nm, about 800-2000 nm, about 900-10000 nm, about 900-5000 nm, about 900-4000 nm, about 1000-5000 nm, about 1000-4000 nm, about 1000-3000 nm or about 1500-3000 nm in diameter or size.

In some instances, the target biological material is retained on the DEP low-field region through an affinity reaction, ionic interactions, electrostatic interactions, direct current (DC) generation or alternating current (AC) generation. In some instances, the target biological material is retained on the DEP intermediate-field region through an affinity reaction, ionic interactions, electrostatic interactions, direct current (DC) generation or alternating current (AC) generation.

Some embodiments provided herein describe methods of isolating target biological material, wherein the method further comprises making the target biological material visualizable. In some embodiments, the method further comprises determining the identity of the target biological material. In additional embodiments, the method further comprises quantifying the amount of the target biological material present. In some embodiments, the method further comprises performing in situ analysis of the target biological sample in the low-field region. In some embodiments, the method further comprises performing in situ analysis of the target biological sample in the intermediate-field region. In certain embodiments, the target biological material is transferred to the high-field region on the array. In some embodiments, non-target biological material is removed by flushing the device with a liquid or buffer. In other embodiments, non-target biological material is selectively degraded in situ on the array. In some instances, the isolated target biological material is collected. In yet other embodiments, the isolated target biological material is further processed. In some embodiments, the target biological material is tested for the presence or absence of one or more biomarkers.

In some embodiments, the target biological material is one or more cellular components. In specific embodiments, the cellular component comprises organelles, mitochondria, apoptotic bodies, endoplasmic reticulum, cell surface membranes, golgi bodies, nuclei, nucleolus, chromosomes, chromatin, nuclear envelope, or combinations thereof. In other embodiments, the target biological material comprises one or more extracellular bodies. In specific embodiments, the extracellular body comprises micelles, large chylomicrons, blood clots, plaques, protein aggregates (e.g. beta-amyloid plaques or tau protein), or combinations thereof. In some embodiments, the target biological material comprises a pathogen. In certain embodiments, the pathogen comprises a bacteria, protist, helminth, nematode, parasite, virus, prion, fungus, or combinations thereof.

Also provided herein in some embodiments are methods, devices, and/or systems for isolating target biological material, wherein the DEP low-field, DEP intermediate-field and DEP high-field regions are produced by an alternating current. In some embodiments, the DEP low-field, DEP intermediate-field and DEP high-field regions are produced using an alternating current having a voltage of 1 volt to 50 volts peak-peak; and/or a frequency of 5 Hz to 5,000,000 Hz and duty cycles from 5% to 50%. In further or additional embodiments, the electrodes are selectively energized to provide the DEP low-field region and subsequently or continuously selectively energized to provide the DEP high-field region. In further or additional embodiments, the electrodes are selectively energized to provide the DEP intermediate-field region. In some embodiments, the electrodes are selectively energized over finite time intervals.

In some embodiments, the samples applied to any of the methods, devices, and/or systems described herein comprise a biological sample (e.g., a body fluid sample), industrial sample, food sample, or environmental sample. In some embodiments, the body fluid sample comprises blood, serum, plasma, urine, sputum, tears, saliva, sweat, mucus, or cerebrospinal fluid (CSF). In some embodiments, wherein the body fluid sample is blood, serum or plasma, the method of isolating the target biological material further comprises isolating intact cells from supernatant in the biological sample, collecting the supernatant, and applying the processed sample (i.e. supernatant) to the device. In some embodiments, the sample applied to the device is substantially free of intact eukaryotic cells.

In other embodiments, the sample applied to any of the methods, devices, and/or systems described herein is an environmental sample. In certain embodiments, the environmental sample is a sample taken from drinking water, a natural body of water, water reservoirs, recreational waters, swimming pools, whirlpools, hot tubs, spas, or water parks. In some embodiments, the sample applied to any of the methods, devices, and/or systems is an industrial sample. In certain embodiments, the industrial sample comprises a pharmaceutical sample, cosmetic sample, clinical sample, chemical reagent, food manufacturing, product manufacturing sample, culture media, innocula, or cleaning solution.

In some embodiments, the target biological material is labeled, dyed or otherwise tagged for later identification and/or tracing prior to its application to any of the methods, devices, and/or systems described herein. In other embodiments, the target biological material is labeled, dyed or tagged after it has been isolated or concentrated. In some embodiments, the sample or target biological material is labeled with a dye comprising SYBR Green I, SYBR Green II, SYBR Gold stains, SYBR DX, Thiazole Organe (TO), SYTO 10, SYTO17, SYTO-13, SYBR14, SYTO-82, TOTO-1, FUN-1, DEAD Red, TO-PRO-1 iodide, TO-PRO-3 iodide, TO-PRO-5-iodide, YOYO-1, YO-PRO-1, BOBO-1, BOBO-3, POPO-1, POPO-3, PicoGreen, ethidium bromide, propidium iodide, acridine orange, 7-aminoactinomycin, hexidium iodide, dihydroethidium, ethidium homodimer, 9-amino-6-chloro-2-methoxyacridine, DAPI, DIPI, indole dye, imidazole dye, actinomycin D, hydroxystilbamine, or combinations thereof. In other embodiments, the sample or target biological material is labeled with a dye comprising acridine, acridine orange, rhodamine, eosin and fluorescein, Coomassie brilliant blue, 1-anilinonaphthalene-8-sulfonate (ANS), 4,4′-bis-1-anilinonaphthalene-8-sulfonate (Bis-ANS), Nile Red, Thioflavin T, Congo Red, 9-(dicyanovinyl)-julolidine (DCVJ), Chrysamine G, fluorescein, dansyl, fluorescamine, rhodamine, o-phthaldialdehyde (OPA), aphthalene-2,3-dicarboxaldehyde (NDA), 6-carboxy-4,5-dichloro-2,7-dimethoxyfluorescein, succinimidyl ester (6-JOE), a protein specific dye, or combinations thereof. In further or additional embodiments, the sample or target biological material is labeled with a dye comprising Safranin-O, toluidine blue, methylene blue, Coomasie brilliant blue, crystal violet, neutral red, Nigrosin, trypan blue, naphthol blue black, merocyanine dyes, 4-[2-N-substituted-1,4-hydropyridin-4-ylidine)ethylidene]cyclohexa-2,5-dien-1-one, red pyrazolone dyes, azomethine dyes, indoaniline dyes, diazamerocyanine dyes, Reichardt's dye, or combinations thereof.

In certain embodiments, the target biological material is detected with at least one antibody or ligand. In some embodiments, the antibody or ligand is labeled with a detection agent. In certain embodiments, the detecting agent comprises colored dyes, fluorescent dyes, chemiluminescent labels, biotinylated labels, radioactive labels, affinity labels, enzyme labels, hapten labels, a metal molecule, quantum dots, or combinations thereof. In some embodiments, the hapten tag comprises fluorophores, myc, nitrotyrosine, biotin, avidin, strepavidin, 2,4-dinitrophenyl, digoxigenin, bromodeoxy uridine, sulfonate, acetylaminoflurene, mercury trintrophonol, or combinations thereof. In some embodiments, the radioactive marker comprises a radioactive isotope including, but not limited to, radioactive isotopes of iodide, cobalt, selenium, hydrogen, carbon, sulfur, phosphorous or combinations thereof. In still other embodiments, the metal moiety comprises gold particles and coated gold particles, which can be converted by silver stains.

In some embodiments, the isolated material comprises greater than about 99%, greater than about 98%, greater than about 95%, greater than about 90%, greater than about 80%, greater than about 70%, greater than about 60%, greater than about 50%, greater than about 40%, greater than about 30%, greater than about 20%, or greater than about 10% of the target biological material by mass. In certain embodiments, the isolated biological target comprises less than about 80%, less than about 70%, less than about 60%, less than about 50%, less than about 40%, less than about 30%, less than about 20%, less than about 10%, less than about 5%, or less than about 2% of non-target biological material by mass.

Some embodiments provided herein describe a method of testing a subject for the presence or absence of a biological material, the method comprising: (a) obtaining a sample from the subject; (b) optionally centrifuging or filtering the sample to separate intact cells from the sample; (c) applying the sample to a device comprising an array of electrodes; (d) creating DEP low-field and DEP high-field regions on the array; (e) selectively retaining material on the DEP low-field region; (f) optionally isolating the retained material; (g) analyzing the retained material; and (h) determining the presence or absence of the biological material. In some embodiments, DEP intermediate-field regions are created on the array. In some embodiments, the subjects are monitored for the presence or absence of the target biological material. In some instances, the presence of the biological material indicates the subject has an increased risk for a disease. In other instances, the absence of the biological material indicates the subject has an increased risk for a disease.

Also described herein in some embodiments is a method of diagnosing a disease in a subject, the method comprising: (a) obtaining a sample from the subject; (b) pre-processing by optionally centrifuging or filtering the sample to separate intact cells from supernatant in the sample; (c) applying the sample to a device comprising an array of electrodes; (d) creating DEP low-field, DEP intermediate-field and DEP high-field regions on the array; (e) selectively retaining the biological material on the DEP low-field region; (f) testing the biological material for the presence of one or more biomarkers; and (g) detecting the presence of one or more biomarkers in the sample, wherein the detection of the biomarker is indicative of the disease. In some instances, the biological material is isolated and collected on the DEP intermediate-field. In some embodiments, the method further comprises using a combination of biomarkers retained in low-field, intermediate-field and high-field DEP regions to make a disease diagnosis.

In some embodiments, the subject is tested for an increased risk or diagnosis of cardiovascular disease, neurodegenerative disease, diabetes, auto-immune disease, inflammatory disease, cancer, metabolic disease, prion disease, pathogenic disease or combinations thereof. The subject can then be administered a therapeutic agent, or combination of therapeutic agents, to prevent or treat the identified condition.

Other embodiments provided herein describe a method of testing an industrial sample for the presence or absence of a biological material, the method comprising: (a) obtaining the industrial sample; (b) applying the sample to a device comprising an array of electrodes; (c) creating DEP low-field, DEP intermediate-field and DEP high-field regions on the array; (d) selectively retaining material on the DEP low-field region; (e) optionally isolating the retained material; (f) analyzing the biological material; and (g) determining the presence or absence of the biological material.

Provided herein in some embodiments is an isolated target biological material that is selectively retained on a DEP low-field region using any of the methods, devices or systems described herein. Also described herein is an isolated target biological material that is selectively retained on a DEP low-field region of an alternating current electrokinetic device. Provided herein in some embodiments is an isolated target biological material that is selectively retained on a DEP intermediate-field region using any of the methods, devices or systems described herein. Also described herein is an isolated target biological material that is selectively retained on a DEP intermediate-field region of an alternating current electrokinetic device.

Some embodiments provided herein describe the use of a target biological material isolated through an alternating current electrokinetic device, or any of the methods, devices or systems described herein, for detecting the presence of one or more biomarkers in a sample (e.g., biological) from which the isolated target biological material has been obtained.

Some embodiments provided herein describe an alternating current electrokinetic device for isolating target biological material from a sample, the device comprising: (a) a housing; and (b) a plurality of alternating current (AC) electrodes within the housing, the AC electrodes configured to be selectively energized to establish dielectrophoretic (DEP) high-field, dielectrophoretic (DEP) intermediate-field and dielectrophoretic (DEP) low-field regions, whereby AC electrokinetic effects provide for separation of the target biological material from other entities in the sample at the DEP low-field region of the device. In some embodiments, AC electrokinetic effects provide for separation of the target biological material from other entities in the sample at the DEP intermediate-field region of the device. In some instances, the surface of the device is in contact or proximal to the electrodes. In some instances, the surface is functionalized with biological ligands that are capable of selectively capturing the target biological material. In some embodiments, the surface selectively captures the target biological material by (a) antibody-antigen interactions; (b) biotin-avidin interactions; (c) ionic or electrostatic interactions; or (d) any combinations thereof. In certain embodiments, the surface comprises one or more magnetic beads in the DEP low-field region. In some embodiments, the magnetic bead is coupled to (a) at least one nucleic acid; (b) at least one antibody; (c) biotin; (d) streptavidin; or (e) any combination thereof. In some embodiments, the surface comprises one or more magnetic beads in the DEP intermediate-field region.

Some embodiments of the device described herein further comprise an electrode at the DEP low-field or intermediate-field region. In certain embodiments, the electrode at the DEP low-field or intermediate-field region functions to retain the target biological material through direct current or alternating current generation. In further or additional embodiments, the device further comprises a well in the DEP low-field or intermediate-field region to retain the target biological material during washing or flushing steps. In further or additional embodiments, the device further comprises a through via in the DEP low-field or intermediate-field region to remove the target biological material to a separate chamber or channel.

Also described herein is a system for isolating target biological material from a sample, the system comprising a device comprising a plurality of alternating current (AC) electrodes within the housing, the AC electrodes configured to be selectively energized to establish dielectrophoretic (DEP) high-field, dielectrophoretic (DEP) intermediate-field and dielectrophoretic (DEP) low-field regions, whereby AC electrokinetic effects provide for separation of the target biological material from other entities in the sample at the DEP low-field or intermediate-field region of the device. In some embodiments, the target biological material is at least 800 nm in diameter.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows isolation of green fluorescent E. coli on an array. Panel (A) shows a bright field view. Panel (B) shows a green fluorescent view of the electrodes before DEP activation. Panel (C) shows E. coli on the electrodes after one minute at 10 kHz, 20 Vp-p in 1×TBE buffer. Panel (D) shows E. coli on the electrodes after one minute at 1 MHz, 20 Vp-p in 1×TBE buffer.

FIG. 2 shows a correlation between AC electrokinetic (ACE) isolation of microparticles in the low-field region vs other markers for myocardial infarction (MI). Qiagen measures total cell-free circulating DNA in the plasma sample. Troponin and CK-MB are cardiac markers for heart damage. None of the parameters correlate with the number of microparticles isolated in the low-field region using ACE.

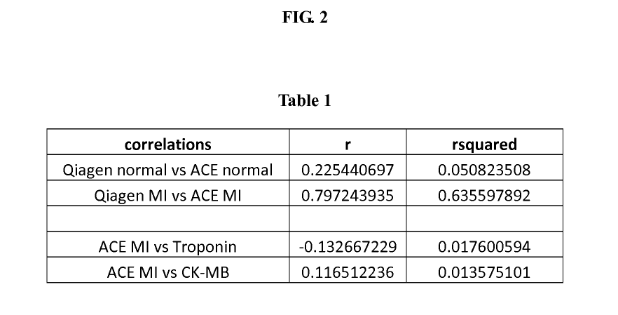

FIG. 3 shows an example of circulating microparticle isolated from healthy and myocardial infarction (MI) patients using AC electrokinetics on an electrode array, detected by SYBR green I staining Results from 3 different normal/healthy plasma donors (control) are displayed at the top row, while results from 3 different MI donors are displayed on the bottom row. Mann Whitney U test of enumerated microparticles in normal (n=11) and MI (n=19) plasma samples was p<0.001, 2-tailed. Ulex Europaeus Agglutinin (UEA) staining yielded no results.

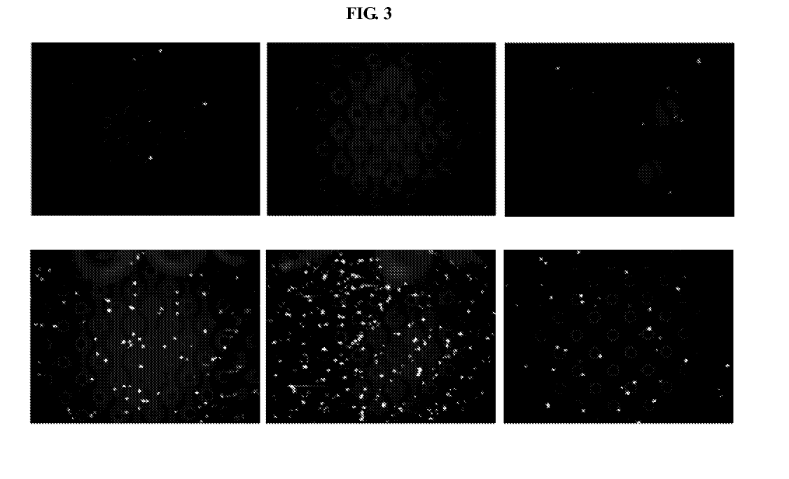

FIG. 4 shows the analysis of AC electrokinetics (ACE) microparticle count isolated at the low-field region for myocardial infarction (MI) patient samples (n=19) and normal or healthy patients (control) samples (n=11). The difference between the two types of samples is highly significant (P<0.001, two-tailed test).

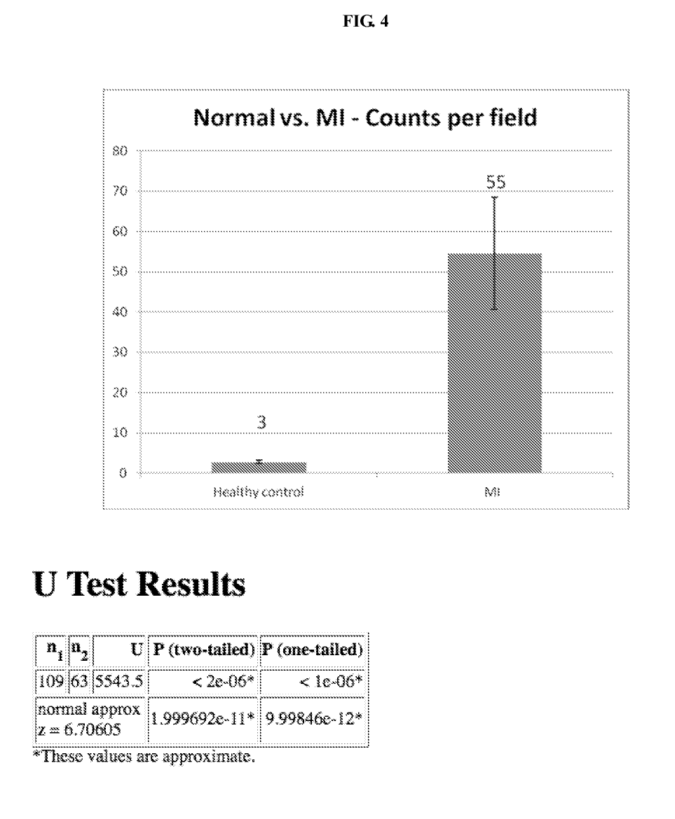

### DETAILED DESCRIPTION OF THE INVENTION

Described herein are methods, devices and systems suitable for isolating or separating particles or molecules from a fluid composition. In specific embodiments, provided herein are methods, devices and systems for isolating or separating target biological material from a sample (e.g., biological sample, industrial sample, food sample, environmental sample, etc.). In various aspects, the methods, devices and systems may allow for a rapid procedure that requires a minimal amount of material and/or results in the isolation of target biological material from complex fluids, such as blood or environmental samples.

Provided in certain embodiments herein are methods, devices and systems for isolating or separating particles or molecules from a fluid composition, the methods, devices, and systems comprising applying the fluid to a device comprising an array of electrodes and being capable of generating alternating (AC) electrokinetic forces (e.g., when the array of electrodes are energized). In some embodiments, the dielectrophoretic field, a component of AC electrokinetic force effects (the others being AC electroosmosis and AC electrothermal effects), comprises low-field regions, intermediate-field regions and/or high-field regions. In specific instances, the particles or molecules are isolated (e.g., isolated or separated from other particles or molecules) in a field region (e.g., a low-field region) of the dielectrophoretic field. In some embodiments, the method, device, or system further includes one or more of the following steps: obtaining samples (e.g., biological), separating intact cells within the biological samples to obtain a eukaryotic cell-free sample, concentrating target biological materials in a first dielectrophoretic field region (e.g., a DEP low-field region), optionally concentrating a target biological material in a first or second dielectrophoretic field region, analyzing the concentrated target biological material, making the target biological material visualizable, determining the identity of the target biological material, and/or quantifying the amount of the target material.

In some embodiments, the method, device, or system further includes one or more of the following steps: concentrating target biological material in a first dielectrophoretic field region (e.g., a DEP low-field region), further concentrating the target biological material in a second dielectrophoretic field region (e.g., a DEP low-field, intermediate-field or high-field region), and washing or flushing away residual material. Optionally, the method also includes devices or systems that are capable of performing one or more of the following steps: washing or otherwise removing intact cells from the starting sample material, washing or rinsing away other materials (e.g., biological) from the target biological material (e.g., rinsing the array with water or buffer while the target biological material is concentrated and maintained within a DEP low-field region of the array), collecting the target biological material, analyzing the concentrated target biological material, making the target biological material visualizable, determining the identity of the target biological material, and/or quantifying the amount of the target material. In some embodiments, the result of the methods, operation of the devices, and operation of the systems described herein is a target biological material, optionally of suitable quantity and purity for later analysis.

In some instances, it is advantageous that the methods described herein are performed in a short amount of time, the devices are operated in a short amount of time, and the systems are operated in a short amount of time. In some embodiments, the period of time is short with reference to the “procedure time” measured from the time between adding the fluid to the device and obtaining an isolated target biological material. In some embodiments, the procedure time is less than 3 hours, less than 2 hours, less than 1 hour, less than 30 minutes, less than 20 minutes, less than 10 minutes, or less than 5 minutes.

In another aspect, the period of time is short with reference to the “hands-on time” measured as the cumulative amount of time that a person must attend to the procedure from the time between adding the fluid to the device and obtaining isolated target biological material. In some embodiments, the hands-on time is less than 20 minutes, less than 10 minutes, less than 5 minute, less than 1 minute, or less than 30 seconds.

In some instances, it is advantageous that the devices described herein comprise a single vessel, the systems described herein comprise a device comprising a single vessel and the methods described herein can be performed in a single vessel, e.g., in a dielectrophoretic device as described herein. In some aspects, such a single-vessel embodiment minimizes the number of fluid handling steps and/or is performed in a short amount of time. In some instances, the present methods, devices and systems are contrasted with methods, devices and systems that use one or more centrifugation steps and/or medium exchanges. In some instances, centrifugation increases the amount of hands-on time required to isolate target biological material. In another aspect, the single-vessel procedure or device isolates target biological material using a minimal amount of consumable reagents.

### Devices and Systems

In one aspect, described herein are devices for isolating and collecting a target biological material from a biological sample. In some embodiments, the device comprises a housing and a reservoir. In some embodiments, the device further comprises a heater. In some embodiments, the heater is capable of increasing the temperature of the fluid to a desired temperature (e.g., about 30° C., 40° C., 50° C., 60° C., 70° C., or the like). In some embodiments, the device further comprises a temperature controller capable of maintaining sub-ambient temperatures (e.g. about 5° C. below ambient, about 10° C. below ambient, about 15° C. below ambient, or the like).

In some embodiments, the device also comprises a plurality of alternating current (AC) electrodes within the housing, the AC electrodes configured to be selectively energized to establish dielectrophoretic (DEP) high-field, dielectrophoretic (DEP) intermediate-field and dielectrophoretic (DEP) low-field regions, whereby AC electrokinetic effects provide for concentration of material (e.g., target biological material) in low-field regions of the device. In some embodiments, AC electrokinetic effects provide for concentration of material (e.g., target biological material) in intermediate-field regions of the device.

In some embodiments, disclosed herein is a device comprising: a. a plurality of alternating current (AC) electrodes, the AC electrodes configured to be selectively energized to establish AC electrokinetic high field, AC electrokinetic intermediate field and AC electrokinetic low field regions; and b. a module capable of thermocycling and performing PCR or other enzymatic reactions. In some embodiments, the plurality of electrodes is configured to be selectively energized to establish a dielectrophoretic high field, dielectrophoretic intermediate field and dielectrophoretic low field regions. In some aspects, the devices disclosed herein are capable of solating biological material from biological samples and/or fluids. In some embodiments, the device is capable of further isolating DNA and performing PCR amplification or other enzymatic reactions. In some embodiments, DNA is isolated and PCR or other enzymatic reaction is performed in a single chamber. In some embodiments, DNA is isolated and PCR or other enzymatic reaction is performed in multiple regions of a single chamber. In some embodiments, DNA is isolated and PCR or other enzymatic reaction is performed in multiple chambers.

In some embodiments, the device further comprises at least one of an elution tube, a chamber and a reservoir to perform PCR amplification or other enzymatic reaction. In some embodiments, PCR amplification or other enzymatic reaction is performed in a serpentine microchannel comprising a plurality of temperature zones. In some embodiments, PCR amplification or other enzymatic reaction is performed in aqueous droplets entrapped in immiscible fluids (i.e., digital PCR). In some embodiments, the thermocycling comprises convection. In some embodiments, the device comprises a surface contacting or proximal to the electrodes, wherein the surface is functionalized with biological ligands that are capable of selectively capturing biomolecules.

For example, further description of the electrodes and the concentration of cells in DEP fields is found in PCT patent publication WO 2009/146143 A2, which is incorporated herein for such disclosure.

In some embodiments, the device comprises a second reservoir comprising an eluent. The eluent is any fluid suitable for eluting the isolated target biological material from the device. In some instances the eluent is water or a buffer. In some instances, the eluent comprises reagents required for further analysis of the target biological material.

In some embodiments, the eluent is a high conductivity buffer. In other embodiments, the eluent is low conductivity buffer. In some embodiments, the eluent has any suitable conductivity. In some embodiments, the conductivity of the eluent is about 1 μS/m. In some embodiments, the conductivity of the eluent is about 5 μS/m. In some embodiments, the conductivity of the eluent is about 10 μS/m. In some embodiments, the conductivity of the eluent is about 15 μS/m. In some embodiments, the conductivity of the eluent is about 100 μS/m. In some embodiments, the conductivity of the eluent is, about 500 μS/m. In some embodiments, the conductivity of the eluent is, about 1 mS/m. In some embodiments, the conductivity of the eluent is about 5 mS/m. In some embodiments, the conductivity of the eluent is, about 10 mS/m. In some embodiments, the conductivity of the eluent is, about 15 mS/m. In some embodiments, the conductivity of the eluent is, about 100 mS/m. In some embodiments, the conductivity of the eluent is about 500 mS/m. In some embodiments, the conductivity of the eluent is about 1 S/m. In some embodiments, the conductivity of the eluent is about 5 S/m. In some embodiments, the conductivity of the eluent is about 10 S/m. In some embodiments, the conductivity is at least 1 μS/m. In some embodiments, the conductivity of the eluent is at least 5 μS/m. In some embodiments, the conductivity of the eluent is at least 10 μS/m. In some embodiments, the conductivity of the eluent is at least 15 μS/m. In some embodiments, the conductivity of the eluent is at least 100 μS/m. In some embodiments, the conductivity of the eluent is, at least 500 μS/m. In some embodiments, the conductivity of the eluent is at least 1 mS/m. In some embodiments, the conductivity of the eluent is at least 5 mS/m. In some embodiments, the conductivity of the eluent is, at least 10 mS/m. In some embodiments, the conductivity of the eluent is at least 15 mS/m. In some embodiments, the conductivity of the eluent is at least 100 mS/m. In some embodiments, the conductivity of the eluent is at least 500 mS/m. In some embodiments, the conductivity of the eluent is at least 1 S/m. In some embodiments, the conductivity of the eluent is at least 5 S/m. In some embodiments, the conductivity of the eluent is at least 10 S/m. In some embodiments, the conductivity is at most 1 μS/m. In some embodiments, the conductivity of the eluent is at most 5 μS/m. In some embodiments, the conductivity of the eluent is at most 10 μS/m. In some embodiments, the conductivity of the eluent is at most 15 μS/m. In some embodiments, the conductivity of the eluent is at most 100 μS/m. In some embodiments, the conductivity of the eluent is at most 500 μS/m. In some embodiments, the conductivity of the eluent is, at most 1 mS/m. In some embodiments, the conductivity of the eluent is at most 5 mS/m. In some embodiments, the conductivity of the eluent is at most 10 mS/m. In some embodiments, the conductivity of the eluent is at most 15 mS/m. In some embodiments, the conductivity of the eluent is at most 100 mS/m. In some embodiments, the conductivity of the eluent is at most 500 mS/m. In some embodiments, the conductivity of the eluent is at most 1 S/m. In some embodiments, the conductivity of the eluent is at most 5 S/m, or at most 10 S/m. In certain embodiments, the conductivity of the eluent is 5 μS/m to 5 S/m.

In some embodiments, chambered devices are created with a variety of pore and/or hole structures (nanoscale, microscale and even macroscale) and contain membranes, gels or filtering materials which control, confine or prevent cells, nanoparticles or other entities (e.g., target biological material, exosomes, non-cell extra cellular bodies, cellular components, microbes, etc.) from diffusing or being transported into the inner chambers while the AC/DC electric fields, solute molecules, buffer and other small molecules can pass through the chambers.

In some embodiments, the device comprises a surface contacting or proximal to the electrodes. In certain embodiments, the surface is functionalized with one or more biological ligands. In some embodiments, the biological ligand is capable of selectively capturing the target biological material. In some embodiments, the functionalized surface captures the target biological material by antibody-antigen interactions; biotin-avidin interactions; biotin-avidin interactions; biotin-streptavidin interactions; ionic interactions; electrostatic interactions; hydrophobic interactions; protein-ligand interactions; protein-antibody interactions or combinations thereof. In some instances, the functionalized surfaces promote retention of the target biological material during subsequent washing or flushing steps.

In some embodiments, the surface comprises one or more magnetic beads. In certain embodiments, the beads are located in or near the DEP low-field region. In certain embodiments, the beads are located in or near the DEP intermediate-field region. In some instances, the magnetic beads are coupled to at least one nucleic acid, at least one antibody, biotin, avidin, streptavidin, or any combination thereof. In some instances, the magnetic beads function to retain the target biological material on the DEP low-field or intermediate-field region during subsequent washing steps.

In some embodiments, the device comprises a well in the DEP low-field region. In some embodiments, the device comprises a well in the DEP intermediate-field region. In certain embodiments, the isolated or retained target biological material remained in or near the well during the washing or flushing steps.

Also provided herein are systems and devices comprising a plurality of alternating current (AC) electrodes, wherein the AC electrodes are configured to be selectively energized to establish dielectrophoretic (DEP) high-field, and dielectrophoretic (DEP) low-field regions. In some embodiments, the AC electrodes are configured to be selectively energized to establish dielectrophoretic (DEP) intermediate-field regions. In some instances, AC electrokinetic effects provide for concentration and/or separation of target biological material in low-field regions and/or concentration (or collection or isolation) of non-target biological material in high-field regions of the DEP field. In some instances, AC electrokinetic effects provide for concentration and/or separation of target biological material in intermediate-field regions. The plurality of alternating current electrodes are optionally configured in any manner suitable for the separation processes described herein. For example, further description of the system or device including electrodes and/or concentration of cells in DEP fields is found in PCT patent publication WO 2009/146143, which is incorporated herein for such disclosure.

In various embodiments these methods, devices and systems are operated in the AC frequency range of from 1,000 Hz to 100 MHz, at voltages which could range from approximately 1 volt to 2000 volts pk-pk; at DC voltages from 1 volt to 1000 volts, at flow rates of from 10 microliters per minute to 10 milliliter per minute, and in temperature ranges from 1° C. to 120° C. In some embodiments, the methods, devices and systems are operated in AC frequency ranges of from about 3 to about 15 kHz. In some embodiments, the methods, devices, and systems are operated at voltages of from 5-25 volts pk-pk. In some embodiments, the methods, devices and systems are operated at voltages of from about 1 to about 50 volts/cm. In some embodiments, the methods, devices and systems are operated at DC voltages of from about 1 to about 5 volts. In some embodiments, the methods, devices and systems are operated at a flow rate of from about 10 microliters to about 500 microliters per minute. In some embodiments, the methods, devices and systems are operated in temperature ranges of from about 20° C. to about 60° C. In some embodiments, the methods, devices and systems are operated in AC frequency ranges of from 1,000 Hz to 10 MHz. In some embodiments, the methods, devices and systems are operated in AC frequency ranges of from 1,000 Hz to 1 MHz. In some embodiments, the methods, devices and systems are operated in AC frequency ranges of from 1,000 Hz to 100 kHz. In some embodiments, the methods, devices and systems are operated in AC frequency ranges of from 1,000 Hz to 10 kHz. In some embodiments, the methods, devices and systems are operated in AC frequency ranges of from 10 kHz to 100 kHz. In some embodiments, the methods, devices and systems are operated in AC frequency ranges of from 100 kHz to 1 MHz. In some embodiments, the methods, devices and systems are operated at voltages from approximately 1 volt to 1500 volts pk-pk. In some embodiments, the methods, devices and systems are operated at voltages from approximately 1 volt to 1500 volts pk-pk. In some embodiments, the methods, devices and systems are operated at voltages from approximately 1 volt to 1000 volts pk-pk. In some embodiments, the methods, devices and systems are operated at voltages from approximately 1 volt to 500 volts pk-pk. In some embodiments, the methods, devices and systems are operated at voltages from approximately 1 volt to 250 volts pk-pk. In some embodiments, the methods, devices and systems are operated at voltages from approximately 1 volt to 100 volts pk-pk. In some embodiments, the methods, devices and systems are operated at voltages from approximately 1 volt to 50 volts pk-pk. In some embodiments, the methods, devices and systems are operated at DC voltages from 1 volt to 1000 volts. In some embodiments, the methods, devices and systems are operated at DC voltages from 1 volt to 500 volts. In some embodiments, the methods, devices and systems are operated at DC voltages from 1 volt to 250 volts. In some embodiments, the methods, devices and systems are operated at DC voltages from 1 volt to 100 volts. In some embodiments, the methods, devices and systems are operated at DC voltages from 1 volt to 50 volts. In some embodiments, the methods, devices, and systems are operated at flow rates of from 10 microliters per minute to 1 ml per minute. In some embodiments, the methods, devices, and systems are operated at flow rates of from 10 microliters per minute to 500 microliters per minute. In some embodiments, the methods, devices, and systems are operated at flow rates of from 10 microliters per minute to 250 microliters per minute. In some embodiments, the methods, devices, and systems are operated at flow rates of from 10 microliters per minute to 100 microliters per minute. In some embodiments, the methods, devices, and systems are operated in temperature ranges from 1° C. to 100° C. In some embodiments, the methods, devices, and systems are operated in temperature ranges from 20° C. to 95° C. In some embodiments, the methods, devices, and systems are operated in temperature ranges from 25° C. to 100° C. In some embodiments, the methods, devices, and systems are operated at room temperature.

In some embodiments, the controller independently controls each of the electrodes. In some embodiments, the controller is externally connected to the device such as by a socket and plug connection, or is integrated with the device housing.

In some embodiments, a system or device described herein comprises a nucleic acid sequencer. The sequencer is optionally any suitable DNA sequencing device including but not limited to a Sanger sequencer, pyro-sequencer, ion semiconductor sequencer, polony sequencer, sequencing by ligation device, DNA nanoball sequencing device, sequencing by ligation device, or single molecule sequencing device.

In some embodiments, a system or device described herein is capable of maintaining a constant temperature. In some embodiments, a system or device described herein is capable of cooling the array or chamber. In some embodiments, a system or device described herein is capable of heating the array or chamber. In some embodiments, a system or device described herein comprises a thermocycler. In some embodiments, the devices disclosed herein comprises a localized temperature control element. In some embodiments, the devices disclosed herein are capable of both sensing and controlling temperature.

In some embodiments, the devices further comprise heating or thermal elements. In some embodiments, a heating or thermal element is localized underneath an electrode. In some embodiments, the heating or thermal elements comprise a metal. In some embodiments, the heating or thermal elements comprise tantalum, aluminum, tungsten, or a combination thereof. Generally, the temperature achieved by a heating or thermal element is proportional to the current running through it. In some embodiments, the devices disclosed herein comprise localized cooling elements. In some embodiments, heat resistant elements are placed directly under the exposed electrode array. In some embodiments, the devices disclosed herein are capable of achieving and maintaining a temperature between about 20° C. and about 120° C. In some embodiments, the devices disclosed herein are capable of achieving and maintaining a temperature between about 30° C. and about 100° C. In other embodiments, the devices disclosed herein are capable of achieving and maintaining a temperature between about 20° C. and about 95° C. In some embodiments, the devices disclosed herein are capable of achieving and maintaining a temperature between about 25° C. and about 90° C., between about 25° C. and about 85° C., between about 25° C. and about 75° C., between about 25° C. and about 65° C. or between about 25° C. and about 55° C. In some embodiments, the devices disclosed herein are capable of achieving and maintaining a temperature of about 20° C., about 30° C., about 40° C., about 50° C., about 60° C., about 70° C., about 80° C., about 90° C., about 100° C., about 110° C. or about 120° C. In some embodiments, the devices disclosed herein surface selectively captures biomolecules on its surface. For example, the devices disclosed herein may capture biomolecules, such as nucleic acids, by, for example, a. nucleic acid hybridization; b. antibody-antigen interactions; c. biotin-avidin interactions; d. ionic or electrostatic interactions; or e. any combination thereof. The devices disclosed herein, therefore, may incorporate a functionalized surface which includes capture molecules, such as complementary nucleic acid probes, antibodies or other protein captures capable of capturing biomolecules (such as nucleic acids), biotin or other anchoring captures capable of capturing complementary target molecules such as avidin, capture molecules capable of capturing biomolecules (such as nucleic acids) by ionic or electrostatic interactions, or any combination thereof.

In some embodiments, the surface is functionalized to minimize and/or inhibit nonspecific binding interactions by: a. polymers (e.g., polyethylene glycol PEG); b. ionic or electrostatic interactions; c. surfactants; or d. any combination thereof. In some embodiments, the methods disclosed herein include use of additives which reduce non-specific binding interactions by interfering in such interactions, such as Tween 20 and the like, bovine serum albumin, nonspecific immunoglobulins, etc.

Array of Electrodes

The plurality of alternating current electrodes are optionally configured in any manner suitable for the separation processes described herein. For example, further description of the system or device including electrodes and/or concentration of cells in DEP fields is found in PCT patent publication WO 2009/146143, which is incorporated herein for such disclosure.

In various embodiments, DEP fields are created or capable of being created by selectively energizing an array of electrodes as described herein. The electrodes are optionally made of any suitable material, including metals (e.g. platinum, palladium, gold, etc.). In various embodiments, electrodes are of any suitable size, of any suitable orientation, of any suitable spacing, energized or capable of being energized in any suitable manner, and the like, such that suitable DEP and/or other electrokinetic fields are produced. In some embodiments, the electrodes can include but are not limited to: aluminum, copper, carbon, iron, silver, gold, palladium, platinum, iridium, platinum iridium alloy, ruthenium, rhodium, osmium, tantalum, titanium, tungsten, polysilicon, and indium tin oxide, or combinations thereof, as well as silicide materials such as platinum silicide, titanium silicide, gold silicide, or tungsten silicide. In some embodiments, the electrodes can comprise a conductive ink capable of being screen-printed.

In some embodiments, the edge to edge (E2E) to diameter ratio of an electrode is about 0.5 mm to about 5 mm. In some embodiments, the E2E to diameter ratio is about 1 mm to about 4 mm. In some embodiments, the E2E to diameter ratio is about 1 mm to about 3 mm. In some embodiments, the E2E to diameter ratio is about 1 mm to about 2 mm. In some embodiments, the E2E to diameter ratio is about 2 mm to about 5 mm. In some embodiments, the E2E to diameter ratio is about 1 mm. In some embodiments, the E2E to diameter ratio is about 2 mm. In some embodiments, the E2E to diameter ratio is about 3 mm. In some embodiments, the E2E to diameter ratio is about 4 mm. In some embodiments, the E2E to diameter ratio is about 5 mm.

In some embodiments, the electrodes disclosed herein are dry-etched. In some embodiments, the electrodes are wet etched. In some embodiments, the electrodes undergo a combination of dry etching and wet etching.

In some embodiments, each electrode is individually site-controlled.

In some embodiments, an array of electrodes is controlled as a unit.

In some embodiments, a passivation layer is employed. In some embodiments, a passivation layer can be formed from any suitable material known in the art. In some embodiments, the passivation layer comprises silicon nitride. In some embodiments, the passivation layer comprises silicon dioxide. In some embodiments, the passivation layer has a relative electrical permittivity of from about 2.0 to about 8.0. In some embodiments, the passivation layer has a relative electrical permittivity of from about 3.0 to about 8.0, about 4.0 to about 8.0 or about 5.0 to about 8.0. In some embodiments, the passivation layer has a relative electrical permittivity of about 2.0 to about 4.0. In some embodiments, the passivation layer has a relative electrical permittivity of from about 2.0 to about 3.0. In some embodiments, the passivation layer has a relative electrical permittivity of about 2.0, about 2.5, about 3.0, about 3.5 or about 4.0.

In some embodiments, the passivation layer is between about 0.1 microns and about 10 microns in thickness. In some embodiments, the passivation layer is between about 0.5 microns and 8 microns in thickness. In some embodiments, the passivation layer is between about 1.0 micron and 5 microns in thickness. In some embodiments, the passivation layer is between about 1.0 micron and 4 microns in thickness. In some embodiments, the passivation layer is between about 1.0 micron and 3 microns in thickness. In some embodiments, the passivation layer is between about 0.25 microns and 2 microns in thickness. In some embodiments, the passivation layer is between about 0.25 microns and 1 micron in thickness.

In some embodiments, the passivation layer is comprised of any suitable insulative low k dielectric material, including but not limited to silicon nitride or silicon dioxide. In some embodiments, the passivation layer is chosen from the group consisting of polyamids, carbon, doped silicon nitride, carbon doped silicon dioxide, fluorine doped silicon nitride, fluorine doped silicon dioxide, porous silicon dioxide, or any combinations thereof. In some embodiments, the passivation layer can comprise a dielectric ink capable of being screen-printed.

### Electrode Geometry

In various embodiments, the electrodes are of any suitable geometry. For example, further description of the system or device including electrodes and/or concentration of cells in DEP fields is found in PCT patent publication WO 2009/146143, which is incorporated herein for such disclosure. In some instances, the electrodes have a curved geometry. In other embodiments, the electrodes have a planar geometry. Other non-limiting examples of suitable electrode geometries include cylindrical, conical, spherical, hemispherical, ellipsoidal, ovoid, or combinations thereof. In certain embodiments, the electrodes have the geometry of two coaxial cylinders or two concentric spheres. In certain embodiments, the electrodes are linear, T-shaped, H-shaped, castellated, and other shapes. In some embodiments, the electrodes are not planar but 3D, wherein the electrodes are vertical, at different z-heights, or combinations thereof.

In some embodiments, the electrodes are in a dot configuration, e.g. the electrodes comprises a generally circular or round configuration. In some embodiments, the angle of orientation between dots is from about 25° to about 60°. In some embodiments, the angle of orientation between dots is from about 30° to about 55°. In some embodiments, the angle of orientation between dots is from about 30° to about 50°. In some embodiments, the angle of orientation between dots is from about 35° to about 45°. In some embodiments, the angle of orientation between dots is about 25°. In some embodiments, the angle of orientation between dots is about 30°. In some embodiments, the angle of orientation between dots is about 35°. In some embodiments, the angle of orientation between dots is about 40°. In some embodiments, the angle of orientation between dots is about 45°. In some embodiments, the angle of orientation between dots is about 50°. In some embodiments, the angle of orientation between dots is about 55°. In some embodiments, the angle of orientation between dots is about 60°.

In some embodiments, the electrodes are in a substantially elongated configuration.

In some embodiments, the electrodes are in a configuration resembling wavy or nonlinear lines. In some embodiments, the array of electrodes is in a wavy or nonlinear line configuration, wherein the configuration comprises a repeating unit comprising the shape of a pair of dots connected by a linker, wherein the dots and linker define the boundaries of the electrode, wherein the linker tapers inward towards or at the midpoint between the pair of dots, wherein the diameters of the dots are the widest points along the length of the repeating unit, wherein the edge to edge distance between a parallel set of repeating units is equidistant, or roughly equidistant. In some embodiments, the electrodes are strips resembling wavy lines, as depicted in FIG. 8. In some embodiments, the edge to edge distance between the electrodes is equidistant, or roughly equidistant throughout the wavy line configuration. In some embodiments, the use of wavy line electrodes, as disclosed herein, lead to an enhanced DEP field gradient.

In some embodiments, the electrodes disclosed herein are in a planar configuration. In some embodiments, the electrodes disclosed herein are in a non-planar configuration.

In some embodiments, the device comprises a plurality of microelectrode devices oriented (a) flat side by side, (b) facing vertically, or (c) facing horizontally. In other embodiments, the electrodes are in a sandwiched configuration, e.g. stacked on top of each other in a vertical format.

In some embodiments described herein are methods, devices and systems in which the electrodes are placed into separate chambers and positive DEP regions and negative DEP regions are created within an inner chamber by passage of the AC DEP field through pore or hole structures. Various geometries are used to form the desired positive DEP (high-field) regions, and DEP negative (low-field) regions for carrying out separations or isolations of material (e.g., target biological material, exosomes, non-cell extra cellular bodies, cellular components, microbes, cells, etc.). In some embodiments, pore or hole structures contain (or are filled with) porous material (hydrogels) or are covered with porous membrane structures. In some embodiments, by segregating the electrodes into separate chambers, such pore/hole structure DEP devices reduce electrochemistry effects, heating, or chaotic fluidic movement from occurring in the inner separation chamber during the DEP process.

In some embodiments, the device further comprises an electrode at the DEP low-field region. In some embodiments, the device further comprises an electrode at the DEP intermediate-field region. In some instances, the electrode at the DEP low-field or intermediate-field region is selectively energized through direct current generation. In other instances, the electrode at the DEP low-field or intermediate-field region is selectively energized through alternating current generation. In some embodiments, the electrode at the DEP low-field or intermediate-field region functions to retain the target biological material at the DEP low-field or intermediate-field region, respectively.

In one aspect, described herein is a device. In some embodiments, electrodes are placed into separate chambers and DEP fields are created within an inner chamber by passage through pore structures. The exemplary device includes a plurality of electrodes and electrode-containing chambers within a housing. A controller of the device independently controls the electrodes, as described further in PCT patent publication WO 2009/146143 A2, which is incorporated herein for such disclosure.

In some embodiments, chambered devices are created with a variety of pore and/or hole structures (nanoscale, microscale and even macroscale) and contain membranes, gels or filtering materials which control, confine or prevent cells, nanoparticles or other entities from diffusing or being transported into the inner chambers while the AC/DC electric fields, solute molecules, buffer and other small molecules can pass through the chambers.

In various embodiments, a variety of configurations for the devices are possible. For example, a device comprising a larger array of electrodes, for example in a square or rectangular pattern configured to create a repeating non-uniform electric field to enable AC electrokinetics. For illustrative purposes only, a suitable electrode array may include, but is not limited to, a 10×10 electrode configuration, a 50×50 electrode configuration, a 10×100 electrode configuration, 20×100 electrode configuration, or a 20×80 electrode configuration.

Such devices include, but are not limited to, multiplexed electrode and chambered devices, devices that allow reconfigurable electric field patterns to be created, devices that combine DC electrophoretic and fluidic processes; sample preparation devices, sample preparation, enzymatic manipulation of isolated nucleic acid molecules and diagnostic devices that include subsequent detection and analysis, lab-on-chip devices, point-of-care and other clinical diagnostic systems or versions.

In some embodiments, a planar platinum electrode array device comprises a housing through which a sample fluid flows. In some embodiments, fluid flows from an inlet end to an outlet end, optionally comprising a lateral analyte outlet. The exemplary device includes multiple AC electrodes. In some embodiments, the sample consists of a combination of micron-sized entities or cells, larger nanoparticulates and smaller nanoparticulates or biomolecules. In some instances, the larger nanoparticulates are cellular debris dispersed in the sample. In some embodiments, the smaller nanoparticulates are proteins, smaller DNA, RNA and cellular fragments. In some embodiments, the planar electrode array device is a 60×20 electrode array that is optionally sectioned into three 20×20 arrays that can be separately controlled but operated simultaneously. The optional auxiliary DC electrodes can be switched on to positive charge, while the optional DC electrodes are switched on to negative charge for electrophoretic purposes. In some instances, each of the controlled AC and DC systems is used in both a continuous and/or pulsed manner (e.g., each can be pulsed on and off at relatively short time intervals) in various embodiments. The optional planar electrode arrays along the sides of the sample flow, when over-layered with nanoporous material (e.g., a hydrogel of synthetic polymer), are optionally used to generate DC electrophoretic forces as well as AC DEP. Additionally, microelectrophoretic separation processes is optionally carried out within the nanopore layers using planar electrodes in the array and/or auxiliary electrodes in the x-y-z dimensions.

In various embodiments, a variety of configurations for the devices are possible (e.g., a device comprising a larger array of electrodes). Such devices include, but are not limited to, multiplexed electrode and chambered devices, devices that allow reconfigurable electric field patterns to be created, devices that combine DC electrophoretic and fluidic processes; sample preparation devices, sample preparation and diagnostic devices that include subsequent detection and analysis, lab-on-chip devices, point-of-care and other clinical diagnostic systems or versions.

In some embodiments, a planar platinum electrode array device comprises a housing through which a sample fluid flows. In some embodiments, fluid flows from an inlet end to an outlet end, optionally comprising a lateral analyte outlet. The exemplary device includes multiple AC electrodes. In some embodiments, the sample consists of a combination of micron-sized entities, larger nanoparticulates and smaller nanoparticulates or biomolecules. In some instances, the larger nanoparticulates are cellular debris dispersed in the sample. In some instances, the micron-sized entities are biomarkers. In some embodiments, the smaller nanoparticulates are proteins, smaller DNA, RNA and cellular fragments. In some embodiments, the planar electrode array device is a 60×20 electrode array that is optionally sectioned into three 20×20 arrays that can be separately controlled but operated simultaneously. The optional auxiliary DC electrodes can be switched on to positive charge, while the optional DC electrodes are switched on to negative charge for electrophoretic purposes. In some instances, each of the controlled AC and DC systems is used in both a continuous and/or pulsed manner (e.g., each can be pulsed on and off at relatively short time intervals) in various embodiments. The optional planar electrode arrays along the sides of the sample flow, when over-layered with nanoporous materials, are optionally used to generate DC electrophoretic forces as well as AC DEP.

In some embodiments, these methods, devices and systems are operated in the AC frequency range of from 5 Hz to 500 mHz. In some embodiments, they are operated in the AC frequency range of from 5 Hz to 400 mHz. In some embodiments, they are operated in the AC frequency range of from 5 Hz to 300 mHz. In some embodiments, they are operated in the AC frequency range of from 5 Hz to 200 mHz. In some embodiments, they are operated in the AC frequency range of from 5 Hz to 100 mHz. In some embodiments, they are operated in the AC frequency range of from 5 Hz to 500 Hz. In some embodiments, they are operated in the AC frequency range of from 5 Hz to 400 Hz. In some embodiments, they are operated in the AC frequency range of from 5 Hz to 300 Hz. In some embodiments, they are operated in the AC frequency range of from 5 Hz to 200 Hz. In some embodiments, they are operated in the AC frequency range of from 5 Hz to 100 Hz. In some embodiments, they are operated in the AC frequency range of from 5 Hz to 75 Hz. In some embodiments, they are operated in the AC frequency range of from, 25 Hz to 500 Hz. In some embodiments, they are operated in the AC frequency range of from, 25 Hz to 400 Hz. In some embodiments, they are operated in the AC frequency range of from 25 Hz to 300 Hz. In some embodiments, they are operated in the AC frequency range of from 25 Hz to 200 Hz. In some embodiments, they are operated in the AC frequency range of from 25 Hz to 150 Hz. In some embodiments, they are operated in the AC frequency range of from 25 Hz to 125 Hz. In some embodiments, they are operated in the AC frequency range of from 50 Hz to 200 Hz. In some embodiments, they are operated in the AC frequency range of from 50 Hz to 150 Hz. In some embodiments, they are operated in the AC frequency range of from 50 Hz to 125 Hz. In some embodiments, they are operated in the AC frequency range of from 75 Hz to 200 Hz. In some embodiments, they are operated in the AC frequency range of from 75 Hz to 150 Hz. In some embodiments, they are operated in the AC frequency range of from 75 Hz to 125 Hz. In some embodiments, they are operated in the AC frequency range of from 100 mHz to 500 mHz. In some embodiments, they are operated in the AC frequency range of from 200 mHz to 500 mHz. In some embodiments, they are operated in the AC frequency range of from 300 mHz to 500 mHz. In some embodiments, they are operated in the AC frequency range of from 300 mHz to 400 mHz. In some embodiments, they are operated in the AC frequency range of from 500 Hz to 200 mHz. In some embodiments, they are operated in the AC frequency range of from 500 mHz to 100 mHz. In some embodiments, they are operated in the AC frequency range of from 500 Hz to 100 mHz. In some embodiments, they are operated in the AC frequency range of from 750 Hz to 100 mHz. In some embodiments, they are operated in the AC frequency range of from 1,000 Hz to 100 mHz. In certain embodiments, the methods devises and systems described herein are operated at the AC frequency range of 5 Hz to 500 mHz. In other embodiments, the methods devices and systems described herein are operated at the AC frequency of 1,000 Hz to 100 mHz. In various embodiments, these methods, devices and systems are operated at voltages which range from approximately 1 volt to 2000 volts pk-pk; at DC voltages from 1 volt to 1000 volts, at flow rates of from 10 microliters per minute to 10 milliliter per minute, and in temperature ranges from 1° C. to 100° C. In some embodiments, the controller independently control each of the electrodes. In some embodiments, the controller is externally connected to the device such as by a socket and plug connection, or is integrated with the device housing.

Some embodiments provided herein describe methods, devices and systems comprising an array of electrodes, wherein the electrodes are selectively energized over finite time intervals. In some embodiments, the electrodes are selectively energized over 5 s. In some embodiments, the electrodes are selectively energized over 10 s. In some embodiments, the electrodes are selectively energized over 15 s. In some embodiments, the electrodes are selectively energized over 20 s. In some embodiments, the electrodes are selectively energized over 30 s. In some embodiments, the electrodes are selectively energized over 45 s. In some embodiments, the electrodes are selectively energized over 60 s. In some embodiments, the electrodes are selectively energized over 1.5 min. In some embodiments, the electrodes are selectively energized over 2 min. In some embodiments, the electrodes are selectively energized over 3 min. In some embodiments, the electrodes are selectively energized over 4 min. In some embodiments, the electrodes are selectively energized over 5 min. In some embodiments, the electrodes are selectively energized over 8 min. In some embodiments, the electrodes are selectively energized over 10 min. In some embodiments, the electrodes are selectively energized over 15 min. In some embodiments, the electrodes are selectively energized over 20 min. In some embodiments, the electrodes are selectively energized over 25 min. In some embodiments, the electrodes are selectively energized over 30 min. In some embodiments, the electrodes are selectively energized over 60 min. In certain embodiments, the electrodes are repeatedly energized for short time periods (e.g., 5 s to 10 min) over several hours or days at finite intervals. In certain embodiments, the electrodes are repeatedly energized for 5 s. In certain embodiments, the electrodes are repeatedly energized for 10 s. In certain embodiments, the electrodes are repeatedly energized for 15 s. In certain embodiments, the electrodes are repeatedly energized for 20 s. In certain embodiments, the electrodes are repeatedly energized for, 30 s. In certain embodiments, the electrodes are repeatedly energized for 45 s. In certain embodiments, the electrodes are repeatedly energized for 60 s. In certain embodiments, the electrodes are repeatedly energized for 1.5 min. In certain embodiments, the electrodes are repeatedly energized for 2 min. In certain embodiments, the electrodes are repeatedly energized for 3 min. In certain embodiments, the electrodes are repeatedly energized for 4 min. In certain embodiments, the electrodes are repeatedly energized for 5 min. In certain embodiments, the electrodes are repeatedly energized for 8 min. In certain embodiments, the electrodes are repeatedly energized for 10 min time periods. In certain embodiments, the electrodes are repeatedly energized over 0.5 h. In certain embodiments, the electrodes are repeatedly energized over 1 h. In certain embodiments, the electrodes are repeatedly energized over 1.5 h. In certain embodiments, the electrodes are repeatedly energized over 2 h. In certain embodiments, the electrodes are repeatedly energized over 2.5 h. In certain embodiments, the electrodes are repeatedly energized over 3 h. In certain embodiments, the electrodes are repeatedly energized over 4 h. In certain embodiments, the electrodes are repeatedly energized over 5 h. In certain embodiments, the electrodes are repeatedly energized over 6 h. In certain embodiments, the electrodes are repeatedly energized over 9 h. In certain embodiments, the electrodes are repeatedly energized over 10 h. In certain embodiments, the electrodes are repeatedly energized over 12 h. In certain embodiments, the electrodes are repeatedly energized over 15 h. In certain embodiments, the electrodes are repeatedly energized over 18 h. In certain embodiments, the electrodes are repeatedly energized over 20 h. In certain embodiments, the electrodes are repeatedly energized over 24 h. In certain embodiments, the electrodes are repeatedly energized over 36 h. In certain embodiments, the electrodes are repeatedly energized over 48 h. In certain embodiments, the electrodes are repeatedly energized over 3 days. In certain embodiments, the electrodes are repeatedly energized over 4 days. In certain embodiments, the electrodes are repeatedly energized over 5 days at finite intervals.

Also described herein are scaled sectioned (x-y dimensional) arrays of robust electrodes and strategically placed (x-y-z dimensional) arrangements of auxiliary electrodes that combine DEP, electrophoretic, and fluidic forces, and use thereof. In some embodiments, clinically relevant volumes of blood, serum, plasma, or other samples are more directly analyzed under higher ionic strength and/or conductance conditions. In other embodiments, the samples are directly analyzed under lower ionic strength and/or conductance conditions. Described herein is the overlaying of robust electrode structures (e.g. platinum, palladium, gold, etc.) with one or more porous layers of materials (natural or synthetic porous hydrogels, membranes, controlled nanopore materials, and thin dielectric layered materials) to reduce the effects of any electrochemistry (electrolysis) reactions, heating, and chaotic fluid movement that may occur on or near the electrodes, and still allow the effective separation of target biological material (e.g., exosomes, non-cell extra cellular bodies, cellular components, microbes such as bacteria and viruses, cells, nanoparticles, DNA, and other biomolecules) to be carried out. In some embodiments, in addition to using AC frequency cross-over points to achieve higher resolution separations, on-device (on-array) DC microelectrophoresis is used for the secondary separations. In some embodiments, the device is sub-sectioned, optionally for purposes of concurrent separations of different materials or entities (e.g., exosomes, non-cell extra cellular bodies, cellular components, microbes such as bacteria and viruses, and DNA) carried out simultaneously on such a device.

### Hydrogels

Overlaying electrode structures with one or more layers of materials can reduce the deleterious electrochemistry effects, including but not limited to electrolysis reactions, heating, and chaotic fluid movement that may occur on or near the electrodes, and still allow the effective separation of cells, bacteria, virus, nanoparticles, DNA, and other biomolecules to be carried out. In some embodiments, the materials layered over the electrode structures may be one or more porous layers. In other embodiments, the one or more porous layers is a polymer layer. In other embodiments, the one or more porous layers is a hydrogel.

In general, the hydrogel should have sufficient mechanical strength and be relatively chemically inert such that it will be able to endure the electrochemical effects at the electrode surface without disconfiguration or decomposition. In general, the hydrogel is sufficiently permeable to small aqueous ions, but keeps biomolecules away from the electrode surface.

In some embodiments, the hydrogel is a single layer, or coating.

In some embodiments, the hydrogel comprises a gradient of porosity, wherein the bottom of the hydrogel layer has greater porosity than the top of the hydrogel layer.

In some embodiments, the hydrogel comprises multiple layers or coatings. In some embodiments, the hydrogel comprises two coats. In some embodiments, the hydrogel comprises three coats. In some embodiments, the bottom (first) coating has greater porosity than subsequent coatings. In some embodiments, the top coat is has less porosity than the first coating. In some embodiments, the top coat has a mean pore diameter that functions as a size cut-off for particles of greater than 100 picometers in diameter.

In some embodiments, the hydrogel has a conductivity from about 0.001 S/m to about 10 S/m. In some embodiments, the hydrogel has a conductivity from about 0.01 S/m to about 10 S/m. In some embodiments, the hydrogel has a conductivity from about 0.1 S/m to about 10 S/m. In some embodiments, the hydrogel has a conductivity from about 1.0 S/m to about 10 S/m. In some embodiments, the hydrogel has a conductivity from about 0.01 S/m to about 5 S/m. In some embodiments, the hydrogel has a conductivity from about 0.01 S/m to about 4 S/m. In some embodiments, the hydrogel has a conductivity from about 0.01 S/m to about 3 S/m. In some embodiments, the hydrogel has a conductivity from about 0.01 S/m to about 2 S/m. In some embodiments, the hydrogel has a conductivity from about 0.1 S/m to about 5 S/m. In some embodiments, the hydrogel has a conductivity from about 0.1 S/m to about 4 S/m. In some embodiments, the hydrogel has a conductivity from about 0.1 S/m to about 3 S/m. In some embodiments, the hydrogel has a conductivity from about 0.1 S/m to about 2 S/m. In some embodiments, the hydrogel has a conductivity from about 0.1 S/m to about 1.5 S/m. In some embodiments, the hydrogel has a conductivity from about 0.1 S/m to about 1.0 S/m.

In some embodiments, the hydrogel has a conductivity of about 0.1 S/m. In some embodiments, the hydrogel has a conductivity of about 0.2 S/m. In some embodiments, the hydrogel has a conductivity of about 0.3 S/m. In some embodiments, the hydrogel has a conductivity of about 0.4 S/m. In some embodiments, the hydrogel has a conductivity of about 0.5 S/m. In some embodiments, the hydrogel has a conductivity of about 0.6 S/m. In some embodiments, the hydrogel has a conductivity of about 0.7 S/m. In some embodiments, the hydrogel has a conductivity of about 0.8 S/m. In some embodiments, the hydrogel has a conductivity of about 0.9 S/m. In some embodiments, the hydrogel has a conductivity of about 1.0 S/m.

In some embodiments, the hydrogel has a thickness from about 0.1 microns to about 10 microns. In some embodiments, the hydrogel has a thickness from about 0.1 microns to about 5 microns. In some embodiments, the hydrogel has a thickness from about 0.1 microns to about 4 microns. In some embodiments, the hydrogel has a thickness from about 0.1 microns to about 3 microns. In some embodiments, the hydrogel has a thickness from about 0.1 microns to about 2 microns. In some embodiments, the hydrogel has a thickness from about 1 micron to about 5 microns. In some embodiments, the hydrogel has a thickness from about 1 micron to about 4 microns. In some embodiments, the hydrogel has a thickness from about 1 micron to about 3 microns. In some embodiments, the hydrogel has a thickness from about 1 micron to about 2 microns. In some embodiments, the hydrogel has a thickness from about 0.5 microns to about 1 micron.

In some embodiments, the viscosity of a hydrogel solution prior to spin-coating ranges from about 0.5 cP to about 5 cP. In some embodiments, a single coating of hydrogel solution has a viscosity of between about 0.75 cP and 5 cP prior to spin-coating. In some embodiments, in a multi-coat hydrogel, the first hydrogel solution has a viscosity from about 0.5 cP to about 1.5 cP prior to spin coating. In some embodiments, the second hydrogel solution has a viscosity from about 1 cP to about 3 cP. The viscosity of the hydrogel solution is based on the polymers concentration (0.1%-10%) and polymers molecular weight (10,000 to 300,000) in the solvent and the starting viscosity of the solvent.

In some embodiments, the first hydrogel coating has a thickness between about 0.5 microns and 1 micron. In some embodiments, the first hydrogel coating has a thickness between about 0.5 microns and 0.75 microns. In some embodiments, the first hydrogel coating has a thickness between about 0.75 and 1 micron. In some embodiments, the second hydrogel coating has a thickness between about 0.2 microns and 0.5 microns. In some embodiments, the second hydrogel coating has a thickness between about 0.2 and 0.4 microns. In some embodiments, the second hydrogel coating has a thickness between about 0.2 and 0.3 microns. In some embodiments, the second hydrogel coating has a thickness between about 0.3 and 0.4 microns.

In some embodiments, the hydrogel comprises any suitable synthetic polymer forming a hydrogel. In general, any sufficiently hydrophilic and polymerizable molecule may be utilized in the production of a synthetic polymer hydrogel for use as disclosed herein. Polymerizable moieties in the monomers may include alkenyl moieties including but not limited to substituted or unsubstituted α,β, unsaturated carbonyls wherein the double bond is directly attached to a carbon which is double bonded to an oxygen and single bonded to another oxygen, nitrogen, sulfur, halogen, or carbon; vinyl, wherein the double bond is singly bonded to an oxygen, nitrogen, halogen, phosphorus or sulfur; allyl, wherein the double bond is singly bonded to a carbon which is bonded to an oxygen, nitrogen, halogen, phosphorus or sulfur; homoallyl, wherein the double bond is singly bonded to a carbon which is singly bonded to another carbon which is then singly bonded to an oxygen, nitrogen, halogen, phosphorus or sulfur; alkynyl moieties wherein a triple bond exists between two carbon atoms. In some embodiments, acryloyl or acrylamido monomers such as acrylates, methacrylates, acrylamides, methacrylamides, etc., are useful for formation of hydrogels as disclosed herein. More preferred acrylamido monomers include acrylamides, N-substituted acrylamides, N-substituted methacrylamides, and methacrylamide. In some embodiments, a hydrogel comprises polymers such as epoxide-based polymers, vinyl-based polymers, allyl-based polymers, homoallyl-based polymers, cyclic anhydride-based polymers, ester-based polymers, ether-based polymers, alkylene-glycol based polymers (e.g., polypropylene glycol), and the like.

In some embodiments, the hydrogel comprises polyhydroxyethylmethacrylate (pHEMA), cellulose acetate, cellulose acetate phthalate, cellulose acetate butyrate, or any appropriate acrylamide or vinyl-based polymer, or a derivative thereof.

In some embodiments, the hydrogel is applied by vapor deposition.

In some embodiments, the hydrogel is polymerized via atom-transfer radical-polymerization via (ATRP).

In some embodiments, the hydrogel is polymerized via reversible addition-fragmentation chain-transfer (RAFT) polymerization.

In some embodiments, additives are added to a hydrogel to increase conductivity of the gel. In some embodiments, hydrogel additives are conductive polymers (e.g., PEDOT: PSS), salts (e.g., copper chloride), metals (e.g., gold), plasticizers (e.g., PEG200, PEG 400, or PEG 600), or co-solvents.

In some embodiments, the hydrogel also comprises compounds or materials which help maintain the stability of the DNA hybrids, including, but not limited to histidine, histidine peptides, polyhistidine, lysine, lysine peptides, and other cationic compounds or substances.

Dielectrophoresis Fields

In some embodiments, the methods, devices and systems described herein provide a mechanism to collect, separate, isolate, detect and/or analyze cells, particles, and/or molecules (such as target biological material) from a fluid material (which optionally contains other materials, such as contaminants, residual cellular material, or the like). For example, dielectrophoresis (DEP) is utilized in various steps of the methods described herein, the devices and systems described herein are capable of generating DEP fields, and the like. In specific embodiments, DEP is utilized to concentrate non-eukaryotic cells, cellular material or organelles and/or other target biological material (e.g., concurrently or at different times). In certain embodiments, methods described herein further comprise energizing the array of electrodes so as to produce the first, second, and any further optional DEP fields. In some embodiments, the devices and systems described herein are capable of being energized so as to produce the first, second, and any further optional DEP fields.

DEP is a phenomenon in which a force is exerted on a dielectric particle when it is subjected to a non-uniform electric field. Depending on the step of the methods described herein, aspects of the devices and systems described herein, and the like, the dielectric particle in various embodiments herein is a biological entity and/or a molecule, such as a target biological material (different steps of the methods described herein or aspects of the devices or systems described herein may be utilized to isolate and separate different components; further, different field regions of the DEP field may be used in different steps of the methods or aspects of the devices and systems described herein). This dielectrophoretic force does not require the particle to be charged in some embodiments. In some instances, the strength of the force depends on the medium and particles' electrical properties, on the particles' shape and size, as well as on the frequency of the electric field. In some instances, fields of a particular frequency selectivity manipulate particles. In certain aspects described herein, these processes allow for the separation of target biological material (e.g., exosomes, non-cell extra cellular bodies, cellular components, microbes such as bacteria and viruses, and DNA) from other components (e.g., in a fluid medium or fluid sample).

In various embodiments provided herein, a method described herein comprises producing a first DEP field region and a second DEP field region with the array. In various embodiments provided herein, a device or system described herein is capable of producing a first DEP field region and a second DEP field region with the array. In some instances, the first and second field regions are part of a single field (e.g., the first and second regions are present at the same time, but are found at different locations within the device and/or upon the array). In some embodiments, the first and second field regions are different fields (e.g. the first region is created by energizing the electrodes at a first time and the second region is created by energizing the electrodes a second time). In specific aspects, the first DEP field region is suitable for concentrating or isolating target biological material (e.g., into a low-field DEP region). In some embodiments, the second DEP field region is suitable for further separating the target biological material from residual material. In some embodiments, the second DEP field region is suitable for concentrating residual material, such as molecules (e.g., nucleic acid) (e.g, into a high-field DEP region). In some instances, a method described herein optionally excludes use of either the first or second DEP field region.

First DEP Field Region

In some aspects, e.g. in high conductance buffers (>100 mS/m), the method described herein comprises applying a sample (e.g., fluid comprising target biological material) to a device comprising an array of electrodes, and, thereby, concentrating the target biological material in a first DEP field region. In some aspects, the devices and systems described herein are capable of applying a sample (e.g., fluid comprising target biological material) to the device comprising an array of electrodes, and, thereby, concentrating the target biological material in a first DEP field region. The first DEP field region is any field region suitable for concentrating target biological material from a fluid. The target biological material is generally concentrated near the array of electrodes. In some embodiments, the first DEP field region is a dielectrophoretic low-field region. In other embodiments, the first DEP field region is a dielectrophoretic intermediate-field region. In some embodiments, the first DEP field region is a dielectrophoretic high-field region.

In some aspects, e.g. low conductance buffers (<100 mS/m), the method described herein comprises applying a sample (e.g., fluid comprising target biological material) to a device comprising an array of electrodes, and, thereby, concentrating the target biological material in a first DEP field region. In some aspects, the devices and systems described herein are capable of applying a sample (e.g., fluid comprising target biological material) to the device comprising an array of electrodes, and concentrating the target biological material in a first DEP field region. In various embodiments, the first DEP field region is any field region suitable for concentrating target biological material from a fluid. In some embodiments, the target biological material is concentrated ON the array of electrodes. In some embodiments, the first DEP field region is a dielectrophoretic low-field region. In some embodiments, the first DEP field region is a dielectrophoretic intermediate-field region. In some embodiments, the first DEP field region is a dielectrophoretic high-field region.

In some aspects, the devices and systems described herein are capable of applying a fluid comprising microparticles or other particulate material to the device comprising an array of electrodes, and concentrating the microparticles in a first DEP field region. In various embodiments, the first DEP field region may be any field region suitable for concentrating microparticles from a fluid. In some embodiments, the microparticles are concentrated on the array of electrodes. In some embodiments, the microparticles are captured in a dielectrophoretic high field region. In some embodiments, the microparticles are captured in a dielectrophoretic intermediate-field region. In some embodiments, the microparticles are captured in a dielectrophoretic low-field region. High, intermediate and low field capture is generally dependent on the conductivity of the fluid, wherein generally, the crossover point is between about 300-500 mS/m.

In some embodiments, the first DEP field region is a dielectrophoretic low field region performed in fluid conductivity of greater than about 300 mS/m. In some embodiments, the first DEP field region is a dielectrophoretic low field region performed in fluid conductivity of less than about 300 mS/m. In some embodiments, the first DEP field region is a dielectrophoretic intermediate field region performed in fluid conductivity of greater than about 300 mS/m. In some embodiments, the first DEP field region is a dielectrophoretic intermediate field region performed in fluid conductivity of less than about 300 mS/m. In some embodiments, the first DEP field region is a dielectrophoretic high field region performed in fluid conductivity of greater than about 300 mS/m. In some embodiments, the first DEP field region is a dielectrophoretic high field region performed in fluid conductivity of less than about 300 mS/m.

In some embodiments, the first DEP field region is a dielectrophoretic low field region performed in fluid conductivity of greater than about 500 mS/m. In some embodiments, the first DEP field region is a dielectrophoretic low field region performed in fluid conductivity of less than about 500 mS/m. In some embodiments, the first DEP field region is a dielectrophoretic intermediate field region performed in fluid conductivity of greater than about 500 mS/m. In some embodiments, the first DEP field region is a dielectrophoretic intermediate field region performed in fluid conductivity of less than about 500 mS/m. In some embodiments, the first DEP field region is a dielectrophoretic high field region performed in fluid conductivity of greater than about 500 mS/m. In some embodiments, the first DEP field region is a dielectrophoretic high field region performed in fluid conductivity of less than about 500 mS/m.

In some embodiments, the first dielectrophoretic field region is produced by an alternating current. The alternating current has any amperage, voltage, frequency, and the like suitable for concentrating target biological material. In some embodiments, the first dielectrophoretic field region is produced using an alternating current having an amperage of 0.1 micro Amperes-10 Amperes; a voltage of 1-50 Volts peak to peak; and/or a frequency of 1-10,000,000 Hz. In some embodiments, the first dielectrophoretic field region is produced using an alternating current having an amperage of 0.1 micro Amperes-10 Amperes; a voltage of 1.5-50 Volts peak to peak; and/or a frequency of 1,000-1,000,000 Hz. In some embodiments, the first DEP field region is produced using an alternating current having a voltage of 5-25 volts peak to peak. In some embodiments, the first DEP field region is produced using an alternating current having a frequency of from 3-15 kHz. In some embodiments, the first DEP field region is produced using an alternating current having an amperage of 1 milliamp to 1 amp. In some embodiments, the first DEP field region is produced using an alternating current having an amperage of 0.1 micro Amperes-1 Ampere. In some embodiments, the first DEP field region is produced using an alternating current having an amperage of 1 micro Amperes-1 Ampere. In some embodiments, the first DEP field region is produced using an alternating current having an amperage of 100 micro Amperes-1 Ampere. In some embodiments, the first DEP field region is produced using an alternating current having an amperage of 500 micro Amperes-500 milli Amperes. In some embodiments, the first DEP field region is produced using an alternating current having a voltage of 1-25 Volts peak to peak. In some embodiments, the first DEP field region is produced using an alternating current having a voltage of 1-10 Volts peak to peak. In some embodiments, the first DEP field region is produced using an alternating current having a voltage of 25-50 Volts peak to peak. In some embodiments, the first DEP field region is produced using a frequency of from 10-1,000,000 Hz. In some embodiments, the first DEP field region is produced using a frequency of from 100-100,000 Hz. In some embodiments, the first DEP field region is produced using a frequency of from 100-10,000 Hz. In some embodiments, the first DEP field region is produced using a frequency of from 10,000-100,000 Hz. In some embodiments, the first DEP field region is produced using a frequency of from 100,000-1,000,000 Hz. In some embodiments, the first DEP field region is produced using a frequency of 500-500,000 Hz, 500-100,000 Hz, 500-50,000 Hz, 500-25,000 Hz, 500-1,000 Hz, 500-1,000,000 Hz, 1,500-1,00,000 Hz, 5,000-1,000,000 Hz, 10,000-1,000,000 Hz, 50,000-1,000,000 Hz, 75,000-1,000,000 Hz, 100,000-1,000,000 Hz, or 500,000-1,000,000 Hz. In certain embodiments, the first DEP field region is produced using a frequency of about 10 KHz. In some embodiments the waveform is a sine wave, square wave, triangle, sawtooth, or a combination thereof.

In some embodiments, the first dielectrophoretic field region is produced with a direct current bias or offset. The direct current has any amperage, voltage, frequency, and the like suitable for concentrating target biological material. In some embodiments, the first dielectrophoretic field region is produced using a direct current having an amperage of 0.1 micro Amperes-1 Amperes; a voltage of 10 milli Volts-10 Volts; and/or a pulse width of 1 milliseconds-1000 seconds and a pulse frequency of 0.001-1000 Hz. In some embodiments, the first DEP field region is produced using a direct current having an amperage of 1 micro Amperes-1 Amperes. In some embodiments, the first DEP field region is produced using a direct current having an amperage of 100 micro Amperes-500 milli Amperes. In some embodiments, the first DEP field region is produced using a direct current having an amperage of 1 milli Amperes-1 Amperes. In some embodiments, the first DEP field region is produced using a direct current having an amperage of 1 micro Amperes-1 milli Amperes. In some embodiments, the first DEP field region is produced using a direct current having a pulse width of 500 milliseconds-500 seconds. In some embodiments, the first DEP field region is produced using a direct current having a pulse width of 500 milliseconds-100 seconds. In some embodiments, the first DEP field region is produced using a direct current having a pulse width of 1 second-1000 seconds. In some embodiments, the first DEP field region is produced using a direct current having a pulse width of 500 milliseconds-1 second. In some embodiments, the first DEP field region is produced using a pulse frequency of 0.01-1000 Hz. In some embodiments, the first DEP field region is produced using a pulse frequency of 0.1-100 Hz. In some embodiments, the first DEP field region is produced using a pulse frequency of 1-100 Hz. In some embodiments, the first DEP field region is produced using a pulse frequency of 100-1000 Hz. In some embodiments, the first DEP field region is produced using a pulse frequency of 0.01-1000 Hz, 0.1-1000 Hz, 1-1000 Hz, 10-1000 Hz, 100-1000 Hz, 500-1000 Hz, 750-1000 Hz, 0.001-750 Hz, 0.001-500 Hz, 0.001-100 Hz, 0.001-10 Hz, 0.001-1 Hz, 1-500 Hz, 1-100 Hz, 10-100 Hz, or 50-550 Hz. In some embodiments, the first DEP field region is produced using a pulse frequency of about 10 KHz.

In some embodiments, a method, device or system described herein is suitable for isolating or separating specific biological material types. In some embodiments, the DEP field of the method, device or system is specifically tuned to allow for the separation or concentration of a specific type of biological material into a field region of the DEP field. In some embodiments, a method, device or system described herein provides more than one field region wherein more than one type of biological material is isolated or concentrated. In some embodiments, a method, device, or system described herein is tunable so as to allow isolation or concentration of different types of biological material within the DEP field regions thereof. In some embodiments, a method provided herein further comprises tuning the DEP field. In some embodiments, a device or system provided herein is capable of having the DEP field tuned. In some instances, such tuning may be in providing a DEP particularly suited for the desired purpose. For example, modifications in the array, the energy, or another parameter are optionally utilized to tune the DEP field. Tuning parameters for finer resolution include electrode diameter, edge to edge distance between electrodes, voltage, frequency, fluid conductivity and hydrogel composition.

In some embodiments, the first DEP field region comprises the entirety of an array of electrodes. In some embodiments, the first DEP field region comprises a portion of an array of electrodes. In some embodiments, the first DEP field region comprises about 90%, about 80%, about 70%, about 60%, about 50%, about 40%, about 30%, about 25%, about 20%, or about 10% of an array of electrodes. In some embodiments, the first DEP field region comprises about a third of an array of electrodes.

Second DEP Field Region

In one aspect, the methods described herein involve concentrating the residual material or non-target biological material in a second DEP field region. In another aspect, the devices and systems described herein are capable of concentrating the residual material or non-target biological material in a second DEP field region. In some embodiments, the second DEP field region is any field region suitable for concentrating residual material or non-target biological material. In some embodiments, the residual material or non-target biological material is concentrated ON the array of electrodes. In some embodiments, the second DEP field region is a dielectrophoretic high-field region. The second DEP field region is, optionally, the same as the first DEP field region.

In some embodiments, the second dielectrophoretic field region is produced by an alternating current. In some embodiments, the alternating current has any amperage, voltage, frequency, and the like suitable for concentrating residual material or non-target biological material. In some embodiments, the alternating current has any amperage, voltage, frequency, and the like suitable for concentrating or retaining the target biological material. In some embodiments, the second dielectrophoretic field region is produced using an alternating current having an amperage of 0.1 micro Amperes-10 Amperes; a voltage of 1-50 Volts peak to peak; and/or a frequency of 1-10,000,000 Hz. In some embodiments, the second dielectrophoretic field region is produced using an alternating current having an amperage of 0.1 micro Amperes-10 Amperes; a voltage of 1.5-50 Volts peak to peak; and/or a frequency of 1,000-1,000,000 Hz. In some embodiments, the second DEP field region is produced using an alternating current having an amperage of 0.1 micro Amperes-1 Ampere. In some embodiments, the second DEP field region is produced using an alternating current having an amperage of 1 micro Amperes-1 Ampere. In some embodiments, the second DEP field region is produced using an alternating current having an amperage of 100 micro Amperes-1 Ampere. In some embodiments, the second DEP field region is produced using an alternating current having an amperage of 500 micro Amperes-500 milli Amperes. In some embodiments, the second DEP field region is produced using an alternating current having a voltage of 1-25 Volts peak to peak. In some embodiments, the second DEP field region is produced using an alternating current having a voltage of 1-10 Volts peak to peak. In some embodiments, the second DEP field region is produced using an alternating current having a voltage of 25-50 Volts peak to peak. In some embodiments, the second DEP field region is produced using a frequency of from 10-1,000,000 Hz. In some embodiments, the second DEP field region is produced using a frequency of from 100-100,000 Hz. In some embodiments, the second DEP field region is produced using a frequency of from 100-10,000 Hz. In some embodiments, the second DEP field region is produced using a frequency of from 10,000-100,000 Hz. In some embodiments, the second DEP field region is produced using a frequency of from 100,000-1,000,000 Hz In some embodiments, the second DEP field region is produced using a frequency of 500-500,000 Hz, 500-100,000 Hz, 500-50,000 Hz, 500-25,000 Hz, 500-1,000 Hz, 500-1,000,000 Hz, 1,500-1,00,000 Hz, 5,000-1,000,000 Hz, 10,000-1,000,000 Hz, 50,000-1,000,000 Hz, 75,000-1,000,000 Hz, 100,000-1,000,000 Hz, or 500,000-1,000,000 Hz.

In some embodiments, the second dielectrophoretic field region is produced with a direct current offset. In some embodiments, the direct current has any amperage, voltage, frequency, and the like suitable for concentrating residual material or non-target biological material. In some embodiments, the direct current has any amperage, voltage, frequency, and the like suitable for concentration or retaining the target biological material. In some embodiments, the second dielectrophoretic field region is produced using a direct current having an amperage of 0.1 micro Amperes-1 Amperes; a voltage of 10 milli Volts-10 Volts; and/or a pulse width of 1 milliseconds-1000 seconds and a pulse frequency of 0.001-1000 Hz. In some embodiments, the second DEP field region is produced using an alternating current having a voltage of 5-25 volts peak to peak. In some embodiments, the second DEP field region is produced using an alternating current having a frequency of from 3-15 kHz. In some embodiments, the second DEP field region is produced using an alternating current having an amperage of 1 milliamp to 1 amp. In some embodiments, the second DEP field region is produced using a direct current having an amperage of 1 micro Amperes-1 Amperes. In some embodiments, the second DEP field region is produced using a direct current having an amperage of 100 micro Amperes-500 milli Amperes. In some embodiments, the second DEP field region is produced using a direct current having an amperage of 1 milli Amperes-1 Amperes. In some embodiments, the second DEP field region is produced using a direct current having an amperage of 1 micro Amperes-1 milli Amperes. In some embodiments, the second DEP field region is produced using a direct current having a pulse width of 500 milliseconds-500 seconds. In some embodiments, the second DEP field region is produced using a direct current having a pulse width of 500 milliseconds-100 seconds. In some embodiments, the second DEP field region is produced using a direct current having a pulse width of 1 second-1000 seconds. In some embodiments, the second DEP field region is produced using a direct current having a pulse width of 500 milliseconds-1 second. In some embodiments, the second DEP field region is produced using a pulse frequency of 0.01-1000 Hz. In some embodiments, the second DEP field region is produced using a pulse frequency of 0.1-100 Hz. In some embodiments, the second DEP field region is produced using a pulse frequency of 1-100 Hz. In some embodiments, the second DEP field region is produced using a pulse frequency of 100-1000 Hz. In some embodiments, the first DEP field region is produced using a pulse frequency of 0.01-1000 Hz, 0.1-1000 Hz, 1-1000 Hz, 10-1000 Hz, 100-1000 Hz, 500-1000 Hz, 750-1000 Hz, 0.001-750 Hz, 0.001-500 Hz, 0.001-100 Hz, 0.001-10 Hz, 0.001-1 Hz, 1-500 Hz, 1-100 Hz, 10-100 Hz, or 50-550 Hz.

In some embodiments, the second DEP field region comprises the entirety of an array of electrodes. In some embodiments, the second DEP field region comprises a portion of an array of electrodes. In some embodiments, the second DEP field region comprises about 90%, about 80%, about 70%, about 60%, about 50%, about 40%, about 30%, about 25%, about 20%, or about 10% of an array of electrodes. In some embodiments, the second DEP field region comprises about a third of an array of electrodes.

### Samples

Some embodiments provided herein describe methods, systems and devices to isolate target biological material from a sample. In one aspect, dielectrophoresis is used to concentrate the target biological material. In some embodiments, the target biological material is contained in a fluid. In some embodiments, the fluid is a liquid, optionally water or an aqueous solution or dispersion.

Certain embodiments provided herein describe methods, systems and devices to isolate target biological material from a biological sample. In some embodiments, the sample is a bodily fluid. Exemplary bodily fluids include blood, serum, plasma, bile, milk, cerebrospinal fluid, gastric juice, ejaculate, mucus, peritoneal fluid, saliva, sweat, tears, urine, sputum, and the like. In some embodiments, target biological material is isolated from bodily fluids using the methods, systems or devices described herein as part of a medical therapeutic or diagnostic procedure, device or system. In some embodiments, the fluid is tissues and/or cells solubilized and/or dispersed in a fluid. In some embodiments, the biological sample is free of intact cells.

Provided herein, in some embodiments, are methods, systems and devices to isolate target biological material from an environmental sample. In some embodiments, the environmental sample is assayed or monitored for the presence of a particular target biological material indicative of a certain contamination, infestation incidence or the like. In some instances, the environmental sample is used to determine the source of a certain contamination, infestation incidence or the like using the methods, devices or systems described herein. Exemplary environmental samples include municipal wastewater, industrial wastewater, a natural body of water, lakes, rivers, oceans, water reservoirs, aquifers, ground water, storm water, plants or portions of plants, animals or portions of animals, insects, municipal water supplies, recreational waters, swimming pools, whirlpools, hot tubs, spas, water parks, drinking water, and the like. Without limitation, this embodiment is beneficial for focusing the target biological material isolation procedure on a particular environmental contaminant, such as a fecal coliform bacterium, whereby DNA sequencing may be used to identify the source of the contaminant.

Also provided herein in some embodiments are methods, systems and devices to isolate target biological material from a food or beverage. In some instances, the food or beverage is assayed or monitored for the presence of a particular target biological material indicative of a certain contamination, infestation incidence or the like. In certain instances, the food or beverage is used to determine the source of a certain contamination, infestation incidence or the like using the methods, devices or systems described herein. Examples of food or beverage samples include but are not limited to beef, pork, sheep, bison, deer, elk, poultry (e.g., chicken and turkey) and fish, produce, juices, dairy products, dry goods (e.g., cereals), and all manners of raw and processed foods.

Also provided herein in some embodiments are methods, systems and devices to isolate target biological material from an industrial sample. Non-limiting examples of an industrial sample include industrial sample comprises a pharmaceutical sample, cosmetic sample, clinical sample, chemical reagent, food sample, product manufacturing sample, culture media, innocula, cleaning solution, and the like.

In some embodiments, the fluid is a culture or growth medium. In some instances, the growth medium is any medium suitable for culturing cells, for example lysogeny broth (LB) for culturing E. coli, Ham's tissue culture medium for culturing mammalian cells, and the like. In some instances, the medium is a rich medium, minimal medium, selective medium, and the like. In some embodiments, the medium comprises or consists essentially of a plurality of clonal cells. In some embodiments, the medium comprises a mixture of at least two species.

In some embodiments, the fluid is water.

In various embodiments, the methods, devices and systems described herein are used with one or more of bodily fluids, environmental samples, and foods and beverages to monitor public health or respond to adverse public health incidences.

The fluid can have any conductivity including a high, intermediate or low conductivity. In some embodiments, the conductivity is between about 1 μS/m to about 10 mS/m. In some embodiments, the conductivity is between about 10 μS/m to about 10 mS/m. In other embodiments, the conductivity is between about 50 μS/m to about 10 mS/m. In yet other embodiments, the conductivity is between about 100 μS/m to about 10 mS/m, between about 100 μS/m to about 8 mS/m, between about 100 μS/m to about 6 mS/m, between about 100 μS/m to about 5 mS/m, between about 100 μS/m to about 4 mS/m, between about 100 μS/m to about 3 mS/m, between about 100 μS/m to about 2 mS/m, or between about 100 μS/m to about 1 mS/m.

In some embodiments, the conductivity is about 1 μS/m. In some embodiments, the conductivity is about 10 μS/m. In some embodiments, the conductivity is about 100 μS/m. In some embodiments, the conductivity is about 1 mS/m. In other embodiments, the conductivity is about 2 mS/m. In some embodiments, the conductivity is about 3 mS/m. In yet other embodiments, the conductivity is about 4 mS/m. In some embodiments, the conductivity is about 5 mS/m. In some embodiments, the conductivity is about 10 mS/m. In still other embodiments, the conductivity is about 100 mS/m. In some embodiments, the conductivity is about 1 S/m. In other embodiments, the conductivity is about 10 S/m.

In some embodiments, the conductivity is at least 1 μS/m. In yet other embodiments, the conductivity is at least 10 μS/m. In some embodiments, the conductivity is at least 100 μS/m. In some embodiments, the conductivity is at least 1 mS/m. In additional embodiments, the conductivity is at least 10 mS/m. In yet other embodiments, the conductivity is at least 100 mS/m. In some embodiments, the conductivity is at least 1 S/m. In some embodiments, the conductivity is at least 10 S/m. In some embodiments, the conductivity is at most 1 μS/m. In some embodiments, the conductivity is at most 10 μS/m. In other embodiments, the conductivity is at most 100 μS/m. In some embodiments, the conductivity is at most 1 mS/m. In some embodiments, the conductivity is at most 10 mS/m. In some embodiments, the conductivity is at most 100 mS/m. In yet other embodiments, the conductivity is at most 1 S/m. In some embodiments, the conductivity is at most 10 S/m.

In some embodiments, the fluid is a small volume of liquid including less than 10 ml. In some embodiments, the fluid is less than 8 ml. In some embodiments, the fluid is less than 5 ml. In some embodiments, the fluid is less than 2 ml. In some embodiments, the fluid is less than 1 ml. In some embodiments, the fluid is less than 500 μl. In some embodiments, the fluid is less than 200 μl. In some embodiments, the fluid is less than 100 μl. In some embodiments, the fluid is less than 50 μl. In some embodiments, the fluid is less than 10 μl. In some embodiments, the fluid is less than 5 μl. In some embodiments, the fluid is less than 1 μl.

In other embodiments, the sample or fluid comprises cells. In various embodiments, the cells are pathogen cells, bacteria cells, plant cells, insect cells, algae cells, cyanobacterial cells, organelles and/or combinations thereof. As used herein, “cells” include viruses. The cells can be microorganisms or cells from multi-cellular organisms. In some instances, the cells comprise a solubilized tissue sample. In various embodiments, the cells are wild-type or genetically engineered. In some instances, the cells comprise a library of mutant cells. In some embodiments, the cells are randomly mutagenized such as having undergone chemical mutagenesis, radiation mutagenesis (e.g. UV radiation), or a combination thereof. In some embodiments, the cells have been transformed with a library of mutant nucleic acid molecules.

In some embodiments, the sample or fluid does not comprise cells. In certain embodiments, sample or fluid does not comprise intact cells. In some embodiments, any of the samples described herein are processed (e.g., spun down or centrifuged) to isolate intact cells from the supernatant. In further or additional embodiments, the supernatant (free from intact cells) is collected and applied to the methods, devices, or systems described herein as the sample. In certain embodiments, the sample applied to the device is substantially free on intact eukaryotic cells.

In some embodiments, the quantity of fluid applied to the device or used in the method comprises less than about 100,000,000 cells. In some embodiments, the fluid comprises less than about 10,000,000 cells. In some embodiments, the fluid comprises less than about 1,000,000 cells. In some embodiments, the fluid comprises less than about 100,000 cells. In some embodiments, the fluid comprises less than about 10,000 cells. In some embodiments, the fluid comprises less than about 1,000 cells.

### Target Biological Material

In some embodiments, the method, device, or system described herein is optionally utilized to obtain, isolate, separate, detect, and/or analyze any desired target biological material that may be obtained from such a method, device or system. Target biological material isolated by the methods, devices and systems described herein include, but are not limited to, non-cell and cellular components, large exosomes, prostasomes, and other non-cell extracellular bodies, cell organelles, microbiological targets, such as bacteria, protists, nematodes, parasites and the like, and combinations thereof.

In some embodiments, the target biological sample is a cellular component. Non-limiting examples of cellular components include organelles, mitochondria, apoptotic bodies, endoplasmic reticulum, cell surface membranes, golgi bodies, nuclei, nucleolus, chromosomes, chromatin, nuclear envelope, and the like. In some embodiments, the target biological sample is an extracellular body. Non-limiting examples of extracellular bodies include micelles, large chylomicrons, blood clots, plaques, protein aggregates (e.g. beta-amyloid plaques or tau protein), and the like. In some instances, extra-cellular DNA (outside cells) is isolated from a sample or fluid. In other embodiments, the target biological sample is a pathogen or component of a pathogen. Examples of pathogens include but are not limited to bacteria, protist, helminth, nematode, parasite, virus, prion, fungus, and the like.

In some embodiments, the target biological material is at least 800 nm in diameter or size. In some embodiments, the target biological materials is at least 900 nm in diameter or size. In some embodiments, the target biological materials is at least 1000 nm in diameter or size. In some embodiments, the target biological materials is at least 1100 nm in diameter or size. In some embodiments, the target biological materials is at least 1200 nm in diameter or size. In some embodiments, the target biological materials is at least 1300 nm in diameter or size. In some embodiments, the target biological materials is at least 1400 nm in diameter or size. In some embodiments, the target biological materials is at least 1500 nm in diameter or size. In some embodiments, the target biological materials is at least 2000 nm in diameter or size. In some embodiments, the target biological materials is at least 2500 nm in diameter or size. In some embodiments, the target biological materials is at least 3000 nm in diameter or size. In some embodiments, the target biological materials is about 800-10000 nm in diameter or size. In some embodiments, the target biological materials is about 800-5000 nm in diameter or size. In some embodiments, the target biological materials is about 800-4000 nm in diameter or size. In some embodiments, the target biological materials is about 800-3000 nm in diameter or size. In some embodiments, the target biological materials is about 800-2000 nm in diameter or size. In some embodiments, the target biological materials is about 900-10000 nm in diameter or size. In some embodiments, the target biological materials is about 900-5000 nm in diameter or size. In some embodiments, the target biological materials is about 900-4000 nm in diameter or size. In some embodiments, the target biological materials is about 1000-5000 nm in diameter or size. In some embodiments, the target biological materials is about 1000-4000 nm in diameter or size. In some embodiments, the target biological materials is about 1000-3000 nm in diameter or size. In some embodiments, the target biological materials is about 1500-3000 nm in diameter or size. In certain embodiments, the target biological material is at least 800 nm. In certain embodiments, the target biological material is at least 900 nm. In certain embodiments, the target biological material is at least 1000 nm. In certain embodiments, the target biological material is at least 1200 nm. In certain embodiments, the target biological material is at least 1500 nm.

In various embodiments, an isolated or separated target biological material is a composition comprising target biological material that is free from at least 99% by mass of other materials, free from at least 99% by mass of residual or non-target materials, free from at least 98% by mass of other materials, free from at least 98% by mass of residual or non-target materials, free from at least 97% by mass of other materials, free from at least 97% by mass of residual or non-target materials, free from at least 96% by mass of other materials, free from at least 96% by mass of residual or non-target materials, free from at least 95% by mass of other materials, free from at least 95% by mass of residual or non-target materials, free from at least 90% by mass of other materials, free from at least 90% by mass of residual or non-target materials, free from at least 80% by mass of other materials, free from at least 80% by mass of residual or non-target materials, free from at least 70% by mass of other materials, free from at least 70% by mass of residual or non-target materials, free from at least 60% by mass of other materials, free from at least 60% by mass of residual or non-target materials, free from at least 50% by mass of other materials, free from at least 50% by mass of residual or non-target materials, free from at least 30% by mass of other materials, free from at least 30% by mass of residual or non-target materials, free from at least 10% by mass of other materials, free from at least 10% by mass of residual or non-target materials, free from at least 5% by mass of other materials, or free from at least 5% by mass of residual or non-target materials.

In various embodiments, the isolated target biological material has any suitable purity. For example, if a downstream analytical procedure can work with samples having about 20% residual cellular material, then isolation of the target biological material to 80% is suitable. In some embodiments, the isolated target biological material comprises less than about 80%, less than about 70%, less than about 60%, less than about 50%, less than about 40%, less than about 30%, less than about 20%, less than about 10%, less than about 5%, or less than about 2% non-target biological material by mass. In some embodiments, the isolated target biological material comprises greater than about 99%, greater than about 98%, greater than about 95%, greater than about 90%, greater than about 80%, greater than about 70%, greater than about 60%, greater than about 50%, greater than about 40%, greater than about 30%, greater than about 20%, or greater than about 10% of the target biological material by mass.

In some embodiments, the target biological material is retained in the device and optionally used in further procedures such as PCR. In some embodiments, the devices and systems are capable of performing PCR or other optional procedures. In other embodiments, the target biological material are collected and/or eluted from the device. In some embodiments, the devices and systems are capable of allowing collection and/or elution of the target biological material from the device or system. In some embodiments, the isolated target biological material are collected by (i) turning off the second dielectrophoretic field region; and (ii) eluting the target biological material from the array in an eluent. Exemplary eluents include water, TE, TBE and L-Histidine buffer.

In some embodiments, the methods described herein result in an isolated target biological material sample that is approximately representative of the starting sample. In some embodiments, the devices and systems described herein are capable of isolating target biological material from a sample that is approximately representative of the starting sample. That is, the population of target biological material collected by the method, or capable of being collected by the device or system, are substantially in proportion to the population of target biological material present in the cells in the fluid.

In some embodiments, the target biological material using the methods described herein or capable of being isolated by the devices described herein is high-quality and/or suitable for using directly in downstream procedures such as DNA sequencing, protein sequencing or PCR.

In some embodiments, the target biological material isolated by the methods described herein or capable of being isolated by the devices described herein has a concentration of at least 0.5 ng/mL. In some embodiments, the target biological material has a concentration of at least 1 ng/mL. In some embodiments, the target biological material has a concentration of at least 5 ng/mL.

In some embodiments, the collected target biological material is further purified using any suitable purification method. Examples of suitable purification methods include chromatography (e.g., pH graded gel chromatography, hydrophobic interaction chromatography, affinity chromatography, immunoaffinity chromatography, immunoprecipitation, ion exchange chromatography, size exclusion chromatography, HPLC, reversed-phase HPLC, etc.), affinity purification, metal binding, 2D-PAGE, filtration, precipitation, ultracentrifugation, centrifugation, or combinations thereof.

### Residual Material

In some embodiments, following concentration of the target biological material in the first DEP field region, the method includes optionally flushing residual non-target material from the device. In some embodiments, the devices or systems described herein are capable of optionally and/or comprising a reservoir comprising a fluid suitable for flushing residual non-target material from the target biological material. In some embodiments, the target biological material is held near the array of electrodes, such as in the first DEP field region, by continuing to energize the electrodes. “Residual material” is anything originally present in the sample or fluid, added during the procedure, created through any step of the process, and the like. For example, residual material includes cell wall fragments, proteins, lipids, carbohydrates, minerals, salts, buffers, plasma, and nucleic acids. It is possible that not all of the target biological material will be concentrated in the first DEP field. In some embodiments, a certain amount of target biological material is flushed with the residual material. In some embodiments, the residual material is retained for other assays (e.g. immunoassays, clinical chemistry, and the like).

In some embodiments, the residual material is flushed in any suitable fluid, for example in water, Tris/Borate/EDTA (TBE) buffer, or the like. In some embodiments, the residual material is flushed with any suitable volume of fluid, flushed for any suitable period of time, flushed with more than one fluid, or any other variation. In some embodiments, the method of flushing residual material is related to the desired level of isolation of the target biological material, with higher purity target biological material requiring more stringent flushing and/or washing. In other embodiments, the method of flushing residual material is related to the particular starting material and its composition. In some instances, a starting material that is high in lipid requires a flushing procedure that involves a hydrophobic fluid suitable for solubilizing lipids.

In some embodiments, the method includes degrading residual material including residual protein and/or nucleic acids. In some embodiments, the devices or systems are capable of degrading residual material including residual protein and/or nucleic acids. For example, proteins are degraded by one or more of chemical degradation (e.g. acid hydrolysis) and enzymatic degradation. In some embodiments, the enzymatic degradation agent is Proteinase K. The optional step of degradation of residual material is performed for any suitable time, temperature, and the like. In some embodiments, the degraded residual material (including degraded proteins) is flushed from the target biological material.

In some embodiments, the agent used to degrade the residual material is inactivated or degraded. In some embodiments, the devices or systems are capable of degrading or inactivating the agent used to degrade the residual material. For example, enzymes including Proteinase K are degraded and/or inactivated using heat (typically, 15 minutes, 70° C.). In some embodiments wherein the residual proteins are degraded by an enzyme, the method further comprises inactivating the enzyme following degradation of the proteins. In some embodiments, heat is provided by a heating module in the device (temp range, e.g., from 30 to 95° C.).

In some instances, the order and/or combination of certain steps of the method is varied. In some embodiments, the devices or methods are capable of performing certain steps in any order or combination. For example, in some embodiments, the residual material and the degraded proteins and/or nucleic acids are flushed in separate or concurrent steps. That is, the residual material is flushed, followed by degradation of residual proteins and/or nucleic acids, followed by flushing degraded proteins and/or nucleic acids from the target biological material. In some embodiments, one first degrades the residual proteins and/or nucleic acids, and then flush both the residual material and degraded proteins and/or nucleic acids from the target biological material in a combined step.

In some embodiments, nucleic acid from the target biological material is retained in the device and optionally used in further procedures such as PCR or other procedures manipulating or amplifying nucleic acid. In some embodiments, the devices and systems are capable of performing PCR or other optional procedures. In other embodiments, the nucleic acids are collected and/or eluted from the device. In some embodiments, the devices and systems are capable of allowing collection and/or elution of nucleic acid from the device or system. In some embodiments, the nucleic acid is collected by (i) turning off the second dielectrophoretic field region; and (ii) eluting the nucleic acid from the array in an eluent. Exemplary eluents include water, TE, TBE and L-Histidine buffer.

### Methods

In one aspect, described herein is a method for isolating a target biological material from a sample or fluid comprising the target biological material. In some embodiments, the method comprises obtaining a sample; applying the sample to a device comprising an array of electrodes, creating DEP low-field, intermediate-field and/or DEP high-field regions on the array, and concentrating the target biological material near a low-field region. In some embodiments, the target biological material is selectively retained on the DEP low-field region of the device through an affinity reaction, ionic interaction, electrostatic interaction, direct current generation, alternating current generation or combinations thereof. In some embodiments, DEP intermediate-field regions are created on the array.

In some instances, residual non-target material is concentrated near the high-field region. In some embodiments, the residual material is washed from the device and/or washed from the target biological material.

In some instances, the target biological material is transferred to a second DEP region (e.g., high-field region). In some instances, the target biological material is collected from the device.

In some embodiments, the target biological material is tested for the presence or absence of one or more biomarkers. In some embodiments, analysis is performed on the target biological material in situ on-chip. In other embodiments, analysis is performed after the target biological material has been eluted from the device for off-chip analysis.

Some embodiments provided herein describe a method of isolating and visualizing a target biological material from a sample. In some embodiments, the target biological material is visualized or detected in the DEP low-field or intermediate-field region of the device. In other embodiments, the target biological material is visualized or detected after being collected from the device. In some embodiments, one or more target biological material is made visualizable or detectable by labeling or dying the target material prior to applying the sample to the devices, methods or systems described herein. In some embodiments, the sample is treated with a dye prior to applying the sample to the devices, methods or systems described herein. In certain embodiments, the isolated target biological material is labeled or dyed. In some instances, the presence or mobility of the target biological material is detected or visualized by virtue of the label, using suitable techniques (e.g., radiographic scanning).

Non-limiting examples of suitable dyes include SYBR Green I, SYBR Green II, SYBR Gold stains, SYBR DX, Thiazole Organe (TO), SYTO 10, SYTO17, SYTO-13, SYBR14, SYTO-82, TOTO-1, FUN-1, DEAD Red, TO-PRO-1 iodide, TO-PRO-3 iodide, TO-PRO-5-iodide, YOYO-1, YO-PRO-1, BOBO-1, BOBO-3, POPO-1, POPO-3, SYPRO orange, SYPRO red, PicoGreen, ethidium bromide, propidium iodide, acridine orange, 7-aminoactinomycin, hexidium iodide, dihydroethidium, ethidium homodimer, 9-amino-6-chloro-2-methoxyacridine, DAPI, DIPI, indole dye, imidazole dye, actinomycin D, hydroxystilbamine, or the like. Other suitable dyes include acridine, acridine orange, rhodamine, eosin and fluorescein, Coomassie brilliant blue, 1-anilinonaphthalene-8-sulfonate (ANS), 4,4′-bis-1-anilinonaphthalene-8-sulfonate (Bis-ANS), Nile Red, Thioflavin T, Congo Red, 9-(dicyanovinyl)-julolidine (DCVJ), Chrysamine G, fluorescein, dansyl, fluorescamine, rhodamine, silver nitrate, o-phthaldialdehyde (OPA), aphthalene-2,3-dicarboxaldehyde (NDA), 6-carboxy-4,5-dichloro-2,7-dimethoxyfluorescein, succinimidyl ester (6-JOE), a protein specific dye, Safranin-O, toluidine blue, methylene blue, crystal violet, neutral red, Nigrosin, trypan blue, naphthol blue black, merocyanine dyes, 4-[2-N-substituted-1,4-hydropyridin-4-ylidine)ethylidene]cyclohexa-2,5-dien-1-one, red pyrazolone dyes, azomethine dyes, indoaniline dyes, diazamerocyanine dyes, Reichardt's dye, or the like.

In some embodiments, the sample is treated with at least one antibody, peptide or ligand prior to applying the sample to the device, wherein the antibody, peptide or ligand specifically binds the target biological material. In some embodiments, the isolated biological material is treated with at least one antibody, peptide or ligand, wherein the antibody, peptide or ligand specifically binds the target biological material. In some embodiments, the antibody, peptide or ligand is labeled with a detection agent. Non-limiting examples of detection agents include colored dyes, fluorescent dyes, fluorescent antibodies, biotinylated antibodies, protein-specific antibodies, chemiluminescent labels, biotinylated labels, radioactive labels, affinity labels, enzyme labels or the like.

Some embodiments provided herein describe a method of determining the identity of the isolated or concentrated target biological material. Other embodiments provided herein describe a method of quantifying the amount of the isolated or concentrated target biological material present in a sample. In some embodiments, the target biological material is identified and/or quantified in the DEP low-field or intermediate-field region of the device. In other embodiments, the target biological material is identified and/or quantified after being collected from the device. In some embodiments, the isolated or concentrated target biological material is analyzed using any suitable method.

Examples of suitable assays for analyzing the target biological material include but are not limited to immunoassays, nucleic acid amplification-based assays, PCR-based assays, nucleic acid hybridization-based assays, bio-sensor assays, immunostaining-microscopy-based assays, nucleic acid-array-based assays, DNA chip-based assays, bacteriophage-detection-based assays, classical microbiology-based assays, and chemical or biochemical assays based on the detection of compounds associated with particular target material, organisms or groups of target organisms, and combinations thereof.

Some embodiments provided herein describe methods of testing a subject for the presence or absence of a biological material, the method comprising obtaining a sample from the subject; optionally centrifuging the sample to separate intact cells from the sample; applying the sample to a device comprising an array of electrodes; creating DEP low-field and DEP high-field regions on the array; selectively retaining material on the DEP low-field region; optionally isolating the retained material; analyzing the retained material; and determining the presence or absence of the biological material. In further or additional embodiments, DEP intermediate-field regions are created on the array and the biological material is retained on the DEP intermediate-field region for analysis.

In some instances, the subject is monitored for the presence or absence of the biological material (e.g., liquid monitoring of tissue damage, monitoring of drug delivery, etc.). In some instances, the presence of the target biological material indicates that the subject has an increased risk for a disease. In other instances, the absence of the target biological material indicates that the subject has an increased risk for a disease. In some embodiments, multi-analyte analysis of DEP high-field, intermediate-field and/or low-field regions is used to monitor the subject.

In some embodiments, provided herein is a method of diagnosing a disease in a subject, wherein the isolated biological material, using any of the methods, systems or devices described herein, is tested for the presence or absence of one or more biomarkers. In some embodiments, the method further comprises detecting the presence of one or more biomarkers in the tested sample, wherein the detection of the biomarker is indicative of the disease. In some embodiments, multi-biomarker analysis of DEP high-field, intermediate-field and/or low-field regions is used to diagnose the subject.

In some embodiments, the methods, devices, or systems described herein are used to treat, diagnose or monitor a disease. In some embodiments, the disease is a cardiovascular disease, neurodegenerative disease, diabetes, auto-immune disease, inflammatory disease, cancer, metabolic disease, prion disease, or pathogenic disease. In some embodiments, the cardiovascular disease is ischemia or an associated injury from reperfusion in the brain, bowel or heart. In some instances, the disease is Ischemic colitis, mesenteric ischemia of the large intestine or small bowel, ischemic stroke, vascular dementia of the brain, angina pectoris, or ischemic heart disease.

Also described herein in some embodiments is a method of testing industrial samples for the presence or absence of a biological material. In some instances, the presence of the target biological material indicates that the tested sample has been contaminated or has degraded. In some instances, the absence of the target biological material indicates that the tested sample has been contaminated or has degraded. In some instances, the presence of the target biological material indicates that the tested sample has not been contaminated or has not degraded. In some instances, the absence of the target biological material indicates that the tested sample has not been contaminated or has not degraded.

Nucleic Acid Assays and Applications

In some embodiments, the methods described herein further comprise optionally isolating and amplifying the nucleic acid isolated from target biological material by polymerase chain reaction (PCR). In some embodiments, the PCR reaction is performed on or near the array of electrodes or in the device. In some embodiments, the device or system comprise a heater and/or temperature control mechanisms suitable for thermocycling.

PCR is optionally done using traditional thermocycling by placing the reaction chemistry analytes in between two efficient thermoconductive elements (e.g., aluminum or silver) and regulating the reaction temperatures using TECs. Additional designs optionally use infrared heating through optically transparent material like glass or thermo polymers. In some instances, designs use smart polymers or smart glass that comprise conductive wiring networked through the substrate. This conductive wiring enables rapid thermal conductivity of the materials and (by applying appropriate DC voltage) provides the required temperature changes and gradients to sustain efficient PCR reactions. In certain instances, heating is applied using resistive chip heaters and other resistive elements that will change temperature rapidly and proportionally to the amount of current passing through them.

In some embodiments, used in conjunction with traditional fluorometry (ccd, pmt, other optical detector, and optical filters), fold amplification is monitored in real-time or on a timed interval. In certain instances, quantification of final fold amplification is reported via optical detection converted to AFU (arbitrary fluorescence units correlated to analyze doubling) or translated to electrical signal via impedance measurement or other electrochemical sensing.

Given the small size of the micro electrode array, these elements are optionally added around the micro electrode array and the PCR reaction will be performed in the main sample processing chamber (over the DEP array) or the analytes to be amplified are optionally transported via fluidics to another chamber within the fluidic cartridge to enable on-cartridge Lab-On-Chip processing.

In some instances, light delivery schemes are utilized to provide the optical excitation and/or emission and/or detection of fold amplification. In certain embodiments, this includes using the flow cell materials (thermal polymers like acrylic (PMMA) cyclic olefin polymer (COP), cyclic olefin co-polymer, (COC), etc.) as optical wave guides to remove the need to use external components. In addition, in some instances light sources-light emitting diodes-LEDs, vertical-cavity surface-emitting lasers—VCSELs, and other lighting schemes are integrated directly inside the flow cell or built directly onto the micro electrode array surface to have internally controlled and powered light sources. Miniature PMTs, CCDs, or CMOS detectors can also be built into the flow cell. This minimization and miniaturization enables compact devices capable of rapid signal delivery and detection while reducing the footprint of similar traditional devices (i.e. a standard bench top PCR/QPCR/Fluorometer).

In some instances, silicon microelectrode arrays can withstand thermal cycling necessary for PCR. In some applications, on-chip PCR is advantageous because small amounts of target nucleic acids can be lost during transfer steps. In certain embodiments of devices, systems or processes described herein, any one or more of multiple PCR techniques are optionally used, such techniques optionally including any one or more of the following: thermal cycling in the flowcell directly; moving the material through microchannels with different temperature zones; and moving volume into a PCR tube that can be amplified on system or transferred to a PCR machine. In some instances, droplet PCR is performed if the outlet contains a T-junction that contains an immiscible fluid and interfacial stabilizers (surfactants, etc). In certain embodiments, droplets are thermal cycled in by any suitable method.

In some embodiments, amplification is performed using an isothermal reaction, for example, transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification or circular helicase-dependent amplification.

In various embodiments, amplification is performed in homogenous solution or as heterogeneous system with anchored primer(s). In some embodiments of the latter, the resulting amplicons are directly linked to the surface for higher degree of multiplex. In some embodiments, the amplicon is denatured to render single stranded products on or near the electrodes. Hybridization reactions are then optionally performed to interrogate the genetic information, such as single nucleotide polymorphisms (SNPs), Short Tandem Repeats (STRs), mutations, insertions/deletions, methylation, etc. Methylation is optionally determined by parallel analysis where one DNA sample is bisulfite treated and one is not. Bisulfite depurinates unmodified C becoming a U. Methylated C is unaffected in some instances. In some embodiments, allele specific base extension is used to report the base of interest.

Rather than specific interactions, the surface is optionally modified with nonspecific moieties for capture. For example, surface could be modified with polycations, i.e., polylysine, to capture DNA molecules which can be released by reverse bias (-V). In some embodiments, modifications to the surface are uniform over the surface or patterned specifically for functionalizing the electrodes or non electrode regions. In certain embodiments, this is accomplished with photolithography, electrochemical activation, spotting, and the like.

In some applications, where multiple chip designs are employed, it is advantageous to have a chip sandwich where the two devices are facing each other, separated by a spacer, to form the flow cell. In various embodiments, devices are run sequentially or in parallel. For sequencing and next generation sequencing (NGS), size fragmentation and selection has ramifications on sequencing efficiency and quality. In some embodiments, multiple chip designs are used to narrow the size range of material collected creating a band pass filter. In some instances, current chip geometry (e.g., 80 um diameter electrodes on 200 um center-center pitch (80/200) acts as 500 bp cutoff filter (e.g., using voltage and frequency conditions around 10 Vpp and 10 kHz). In such instances, a nucleic acid of greater than 500 bp is captured, and a nucleic acid of less than 500 bp is not. Alternate electrode diameter and pitch geometries have different cutoff sizes such that a combination of chips should provide a desired fragment size. In some instances, a 40 um diameter electrode on 100 um center-center pitch (40/100) has a lower cutoff threshold, whereas a 160 um diameter electrode on 400 um center-center pitch (160/400) has a higher cutoff threshold relative to the 80/200 geometry, under similar conditions. In various embodiments, geometries on a single chip or multiple chips are combined to select for a specific sized fragments or particles. For example a 600 bp cutoff chip would leave a nucleic acid of less than 600 bp in solution, then that material is optionally recaptured with a 500 bp cutoff chip (which is opposing the 600 bp chip). This leaves a nucleic acid population comprising 500-600 bp in solution. This population is then optionally amplified in the same chamber, a side chamber, or any other configuration. In some embodiments, size selection is accomplished using a single electrode geometry, wherein nucleic acid of >500 bp is isolated on the electrodes, followed by washing, followed by reduction of the ACE high field strength (change voltage, frequency, conductivity) in order to release nucleic acids of <600 bp, resulting in a supernatant nucleic acid population between 500-600 bp.

In some embodiments, the chip device is oriented vertically with a heater at the bottom edge which creates a temperature gradient column. In certain instances, the bottom is at denaturing temperature, the middle at annealing temperature, the top at extension temperature. In some instances, convection continually drives the process. In some embodiments, provided herein are methods or systems comprising an electrode design that specifically provides for electrothermal flows and acceleration of the process. In some embodiments, such design is optionally on the same device or on a separate device positioned appropriately. In some instances, active or passive cooling at the top, via fins or fans, or the like. provides a steep temperature gradient. In some instances the device or system described herein comprises, or a method described herein uses, temperature sensors on the device or in the reaction chamber monitor temperature and such sensors are optionally used to adjust temperature on a feedback basis. In some instances, such sensors are coupled with materials possessing different thermal transfer properties to create continuous and/or discontinuous gradient profiles.

In some embodiments, the amplification proceeds at a constant temperature (i.e, isothermal amplification).

In some embodiments, the methods disclosed herein further comprise sequencing the nucleic acid isolated as disclosed herein. In some embodiments, the nucleic acid is sequenced by Sanger sequencing or next generation sequencing (NGS). In some embodiments, the next generation sequencing methods include, but are not limited to, pyrosequencing, ion semiconductor sequencing, polony sequencing, sequencing by ligation, DNA nanoball sequencing, sequencing by ligation, or single molecule sequencing.

In some embodiments, the isolated nucleic acids disclosed herein are used in Sanger sequencing. In some embodiments, Sanger sequencing is performed within the same device as the nucleic acid isolation (Lab-on-Chip). Lab-on-Chip workflow for sample prep and Sanger sequencing results would incorporate the following steps: a) sample extraction using ACE chips; b) performing amplification of target sequences on chip; c) capture PCR products by ACE; d) perform cycle sequencing to enrich target strand; e) capture enriched target strands; f) perform Sanger chain termination reactions; perform electrophoretic separation of target sequences by capillary electrophoresis with on chip multi-color fluorescence detection. Washing nucleic acids, adding reagent, and turning off voltage is performed as necessary. Reactions can be performed on a single chip with plurality of capture zones or on separate chips and/or reaction chambers.

In some embodiments, the method disclosed herein further comprise performing a reaction on the nucleic acids (e.g., fragmentation, restriction digestion, ligation of DNA or RNA). In some embodiments, the reaction occurs on or near the array or in a device, as disclosed herein.

The isolated nucleic acids disclosed herein may be further utilized in a variety of assay formats. For instance, devices which are addressed with nucleic acid probes or amplicons may be utilized in dot blot or reverse dot blot analyses, base-stacking single nucleotide polymorphism (SNP) analysis, SNP analysis with electronic stringency, or in STR analysis. In addition, such devices disclosed herein may be utilized in formats for enzymatic nucleic acid modification, or protein-nucleic acid interaction, such as, e.g., gene expression analysis with enzymatic reporting, anchored nucleic acid amplification, or other nucleic acid modifications suitable for solid-phase formats including restriction endonuclease cleavage, endo- or exo-nuclease cleavage, minor groove binding protein assays, terminal transferase reactions, polynucleotide kinase or phosphatase reactions, ligase reactions, topoisomerase reactions, and other nucleic acid binding or modifying protein reactions.

In addition, the devices disclosed herein can be useful in immunoassays. For instance, in some embodiments, locations of the devices can be linked with antigens (e.g., peptides, proteins, carbohydrates, lipids, proteoglycans, glycoproteins, etc.) in order to assay for antibodies in a bodily fluid sample by sandwich assay, competitive assay, or other formats. Alternatively, the locations of the device may be addressed with antibodies, in order to detect antigens in a sample by sandwich assay, competitive assay, or other assay formats. As the isoelectric point of antibodies and proteins can be determined fairly easily by experimentation or pH/charge computations, the electronic addressing and electronic concentration advantages of the devices may be utilized by simply adjusting the pH of the buffer so that the addressed or analyte species will be charged.

In some embodiments, the isolated nucleic acids are useful for use in immunoassay-type arrays or nucleic acid arrays.

### DEFINITIONS AND ABBREVIATIONS

The articles “a”, “an” and “the” are non-limiting. For example, “the method” includes the broadest definition of the meaning of the phrase, which can be more than one method.

“Vp-p” is the peak-to-peak voltage.

“DEP” is an abbreviation for dielectrophoresis.

TBE” is a buffer solution containing a mixture of Tris base, boric acid and EDTA.

“CK-MB” is a cardiac enzyme, creatine kinase MB.

“ACE” is an abbreviation for AC Electrokinetic.

“MI” is an abbreviation for myocardial infarction.

### Examples

### Example 1

### Manipulation of Microparticles by AC Electrokinetics

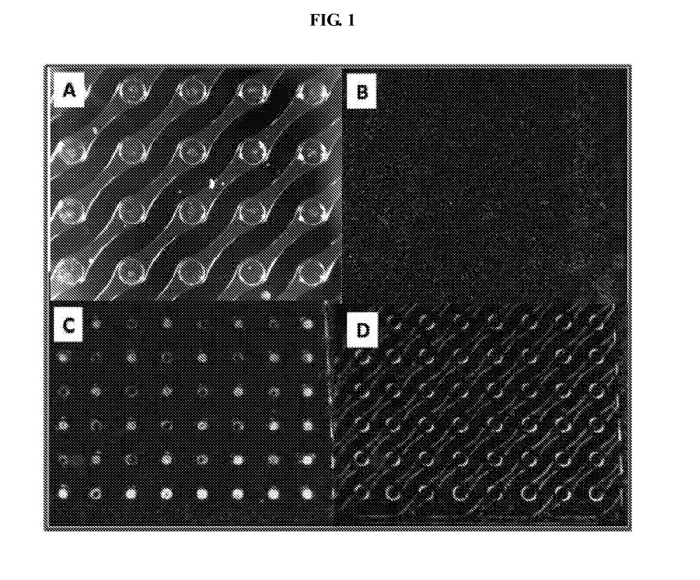

AC electrokinetic parameters are changed to alter the force field experienced by microparticles and cause isolation on high-, intermediate- or low-field regions of the electrode array (FIG. 1).

Suspension containing E. coli in 1×TBE was dispensed onto a chip and AC electrokinetic isolation parameters were applied (20 Vpp, 10 KHz sine, 1 min). Bacteria were collected on the electrodes in the high-field region (FIG. 1c).

Changing the frequency to 1 MHz (20 Vpp, 1 min) caused the bacteria to move away from the high-field region and toward a lower field region (not at the minimal field region) (FIG. 1d). A wider frequency and/or voltage sweep moved microparticles to the low-field region.

This example illustrates that AC electrokinetic parameters affect where the microparticles collect and that there are intermediate-field zones between the high- and low-field regions.

### Example 2

### Ischemia Diagnosis and Monitoring

Isolation of biological material from a biological sample is used for detection or diagnosis of ischemia or an associated injury from reperfusion in the brain, bowel or heart.

Ischemic colitis, mesenteric ischemia of the large intestine or small bowel, ischemic stroke, vascular dementia of the brain, angina pectoris, ischemic heart disease are diagnosed and monitored by analysis of the isolated biological material. Blood, plasma or serum samples are taken from patients showing signs of distress associated with ischemia. The samples are optionally processed to produce samples free from intact cells. The samples are dispensed onto a chip and AC electrokinetic isolation parameters are applied. Circulating microparticles are isolated on the electrodes in the low-field region. In some instances, the microparticles are detected and analyzed in situ on-chip. In other instances, the microparticles are eluted with a liquid, then detected and analyzed off-chip. The microparticles detection and analysis provides earlier diagnosis and treatment of the disease or disorder. The absence, presence, or amount of the microparticles is monitored to follow the progression of the disease or disorder.

### Example 3

### Cardiovascular Event Diagnosis and Monitoring

Plasma samples were collected from myocardial infarction (MI) patients. Plasma samples were also collected from healthy individuals. Sybr Green 1 dye was added directly to the plasma samples from MI patients and healthy individuals at 5× the recommended concentration and incubated for 5′ mins at room temperature.

A fifty microliter aliquot of each sample was dispensed onto a chip and AC Electrokinetic isolation parameters were applied (7 Vpp, 10 KHz sine, 15 min). Microparticles were isolated on the electrodes at the low-field region. Microparticles did not collect in the high-field region or the intermediate-field region. The microparticles at the low-field region were washed with approximately 50 μL TE buffer with electrodes energized.

Images of circulating microparticles on the chip were taken using FITC fluorescence excitation. The microparticles were stained with SYBR indicating that DNA was associated with the fragments. The microparticles were enumerated in MI and healthy plasma. The number of microparticles in MI samples and healthy donors were compared by Mann-Whitney U rank sum statistics. Results indicated a highly significant difference between populations (p<1e-5). Secondary analysis of the microparticles was demonstrated by eluting the target material (microparticles) and performing PCR on the isolate. Primers for GAPDH sequences were used in qPCR.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for isolating a target biological material from a sample, the method comprising:
a. applying the sample to a device, the device comprising an array of electrodes;
b. creating dielectrophoretic (DEP) low-field and dielectrophoretic (DEP) high-field regions on the array; and
c. selectively retaining the target biological material on the DEP low-field region.

2. The method of claim 1, wherein the target biological material is at least 800 nm, at least 900 nm, at least 1000 nm, at least 1100, at least 1200, at least 1300, at least 1400, at least 1500 nm, at least 2000 nm, at least 2500 nm, at least 3000, about 800-10000 nm, about 800-5000 nm, about 800-4000 nm, about 800-3000 nm, about 800-2000 nm, about 900-10000 nm, about 900-5000 nm, about 900-4000 nm, about 1000-5000 nm, about 1000-4000 nm, about 1000-3000 nm or about 1500-3000 nm in diameter or size.

3. The method of any of the preceding claims, wherein the target biological material is retained on the DEP low-field region through an affinity reaction, ionic interactions, electrostatic interactions, direct current generation or alternating current generation.

4. The method of any of the preceding claims, wherein the target biological material is made visualizable.

5. The method of any of the preceding claims, further comprising determining the identity of the target biological material.

6. The method of any of the preceding claims, further comprising quantifying the amount of the target biological material present.

7. The method of any of the preceding claims, further comprising performing in situ analysis of the target biological sample in the low-field region.

8. The method of any of the preceding claims, further comprising collecting said target biological material.

9. The method of any of the preceding claims, further comprising transferring said target biological material to the high-field region on the array.

10. The method of any of the preceding claims, further comprising testing the target biological material for the presence of one or more biomarkers.

11. The method of any of the preceding claims, wherein the target biological material comprises one or more cellular components.

12. The method of claim 11, wherein the cellular component comprises organelles, mitochondria, apoptotic bodies, endoplasmic reticulum, cell surface membranes, golgi bodies, nuclei, nucleolus, chromosomes, chromatin, nuclear envelope, or combinations thereof.

13. The method of any of the preceding claims, wherein the target biological material comprises one or more extracellular bodies.

14. The method of claim 13, wherein the extracellular body comprises micelles, large chylomicrons, blood clots, plaques, protein aggregates (e.g. beta-amyloid plaques or tau protein), or combinations thereof.

15. The method of any of the preceding claims, wherein the target biological material comprises a pathogen.

16. The method of claim 15, wherein the pathogen comprises a bacteria, protist, helminth, nematode, parasite, virus, prion, fungus, or combinations thereof.

17. The method of any of the preceding claims, wherein the DEP low-field and DEP high-field regions are produced by an alternating current.

18. The method of any of the preceding claims, wherein the DEP low-field and DEP high-field regions are produced using an alternating current having a voltage of 1 volt to 50 volts peak-peak; and/or a frequency of 5 Hz to 5,000,000 Hz and duty cycles from 5% to 50%.

19. The method of any of the preceding claims, wherein the electrodes are selectively energized to provide the DEP low-field region and subsequently or continuously selectively energized to provide the DEP high-field region.

20. The method of any of the preceding claims, wherein the sample comprises a body fluid sample, industrial sample, food sample, or environmental sample.

21. The method of claim 20, wherein the body fluid sample comprises blood, serum, plasma, urine, sputum, tears, saliva, sweat, mucus, or cerebrospinal fluid (CSF).

22. The method of claim 20, wherein the body fluid sample is blood, serum, or plasma.

23. The method of claim 22, further comprising
a. isolating intact cells from supernatant; and
b. collecting the supernatant and applying the sample (i.e. supernatant) to the device, wherein the sample applied to the device is substantially free of intact eukaryotic cells.

24. The method of claim 20, wherein the environmental sample is a sample taken from drinking water, a natural body of water, water reservoirs, recreational waters, swimming pools, whirlpools, hot tubs, spas, or water parks.

25. The method of claim 20, wherein the industrial sample comprises a pharmaceutical sample, cosmetic sample, clinical sample, chemical reagent, culture media, innocula, or cleaning solution.

26. The method of any of the preceding claims wherein the electrodes are selectively energized over finite time intervals.

27. The method of any of the preceding claims, further comprising labeling the sample prior to applying the sample to the device.

28. The method of any of the preceding claims, further comprising labeling the isolated target biological material.

29. The method of any one of claims 27-28, wherein the sample or target biological material is labeled with a dye comprising SYBR Green I, SYBR Green II, SYBR Gold stains, SYBR DX, Thiazole Organe (TO), SYTO 10, SYTO17, SYTO-13, SYBR14, SYTO-82, TOTO-1, FUN-1, DEAD Red, TO-PRO-1 iodide, TO-PRO-3 iodide, TO-PRO-5-iodide, YOYO-1, YO-PRO-1, BOBO-1, BOBO-3, POPO-1, POPO-3, PicoGreen, ethidium bromide, propidium iodide, acridine orange, 7-aminoactinomycin, hexidium iodide, dihydroethidium, ethidium homodimer, 9-amino-6-chloro-2-methoxyacridine, DAPI, DIPI, indole dye, imidazole dye, actinomycin D, hydroxystilbamine, or combinations thereof.

30. The method of any one of claims 27-28, wherein the sample or target biological material is labeled with a dye comprising acridine, acridine orange, rhodamine, eosin and fluorescein, Coomassie brilliant blue, 1-anilinonaphthalene-8-sulfonate (ANS), 4,4′-bis-1-anilinonaphthalene-8-sulfonate (Bis-ANS), Nile Red, Thioflavin T, Congo Red, 9-(dicyanovinyl)-julolidine (DCVJ), Chrysamine G, fluorescein, dansyl, fluorescamine, rhodamine, o-phthaldialdehyde (OPA), aphthalene-2,3-dicarboxaldehyde (NDA), 6-carboxy-4,5-dichloro-2,7-dimethoxyfluorescein, succinimidyl ester (6-JOE), a protein specific dye, or combinations thereof.

31. The method of any one of claims 27-28, wherein the sample or target biological material is labeled with a dye comprising Safranin-O, toluidine blue, methylene blue, crystal violet, neutral red, Nigrosin, trypan blue, naphthol blue black, merocyanine dyes, 4-[2-N-substituted-1,4-hydropyridin-4-ylidine)ethylidene]cyclohexa-2,5-dien-1-one, red pyrazolone dyes, azomethine dyes, indoaniline dyes, diazamerocyanine dyes, Reichardt's dye, or combinations thereof.

32. The method of any of the preceding claims, further comprising the step of detecting the target biological material with at least one antibody or ligand.

33. The method of claim 32, wherein the antibody or ligand is labeled with a detection agent.

34. The method of claim 33, wherein the detecting agent comprises colored dyes, fluorescent dyes, chemiluminescent labels, biotinylated labels, radioactive labels, affinity labels, enzyme labels or combinations thereof.

35. The method of any of the preceding claims, wherein the isolated material comprises greater than about 99%, greater than about 98%, greater than about 95%, greater than about 90%, greater than about 80%, greater than about 70%, greater than about 60%, greater than about 50%, greater than about 40%, greater than about 30%, greater than about 20%, or greater than about 10% of the target biological material by mass.

36. The method of any of the preceding claims, wherein non-target biological material is removed by flushing the device with a liquid or buffer.

37. The method of any of the preceding claims, wherein the isolated biological target comprises less than about 80%, less than about 70%, less than about 60%, less than about 50%, less than about 40%, less than about 30%, less than about 20%, less than about 10%, less than about 5%, or less than about 2% of non-target biological material by mass.

38. A method of testing a subject for the presence or absence of a biological material, the method comprising:
a. obtaining a sample from the subject;
b. optionally centrifuging the sample to separate intact cells from the sample;
c. applying the sample to a device comprising an array of electrodes;
d. creating DEP low-field and DEP high-field regions on the array;
e. selectively retaining material on the DEP low-field region;
f. optionally isolating the retained material;
g. analyzing the retained material; and
h. determining the presence or absence of the biological material.

39. The method of claim 38, further comprising monitoring the subject for the presence or absence of the biological material.

40. The method of claim 38, wherein the presence of the biological material indicates the subject has an increased risk for a disease.

41. The method of claim 40, wherein the disease is a cardiovascular disease, neurodegenerative disease, diabetes, auto-immune disease, inflammatory disease, cancer, metabolic disease, prion disease, or pathogenic disease.

42. A method of testing an industrial sample for the presence or absence of a biological material, the method comprising:
a. obtaining the industrial sample;
b. applying the sample to a device comprising an array of electrodes;
c. creating DEP low-field and DEP high-field regions on the array;
d. selectively retaining material on the DEP low-field region;
e. optionally isolating the retained material;
f. analyzing the biological material; and
g. determining the presence or absence of the biological material.

43. A method of diagnosing a disease in a subject, the method comprising:
a. obtaining a sample from the subject;
b. optionally centrifuging the sample to separate cells from the sample;
c. applying the sample to a device comprising an array of electrodes;
d. creating DEP low-field and DEP high-field regions on the array;
e. selectively retaining the biological material on the DEP low-field region;
f. testing the biological material for the presence of one or more biomarkers; and
g. detecting the presence of one or more biomarkers in the sample, wherein the detection of the biomarker is indicative of the disease.

44. The method of claim 43, wherein the disease is a cardiovascular disease, neurodegenerative disease, diabetes, auto-immune disease, inflammatory disease, cancer, metabolic disease, prion disease, or pathogenic disease

45. The isolated biological material of any one of claims 1-44 that is selectively retained on the DEP low-field region of the device.

46. An alternating current electrokinetic device for isolating target biological material from a sample, the device comprising:
a. a housing
b. a plurality of alternating current (AC) electrodes within the housing, the AC electrodes configured o be selectively energized to establish dielectrophoretic (DEP) high-field and dielectrophoretic (DEP) low-field regions, whereby AC electrokinetic effects provide for separation of the target biological material from other entities in the sample at the DEP low-field region of the device.

47. The device of claim 46, wherein the device comprises a surface contacting or proximal to the electrodes.

48. The device of claim 47, wherein the surface is functionalized with biological ligands that are capable of selectively capturing the target biological material.

49. The device of claim 48, wherein the surface selectively captures the target biological material by
a. antibody-antigen interactions;
b. biotin-avidin interactions;
c. ionic or electrostatic interactions; or
d. any combinations thereof.

50. The device of claim 47, wherein the surface comprises one or more magnetic beads in the DEP low-field region.

51. The device of claim 50, wherein the magnetic bead is coupled to
a. at least one nucleic acid;
b. at least one antibody;
c. biotin;
d. streptavidin; or
e. any combination thereof.

52. The device of any one of claim 46-51, further comprising an electrode at the DEP low-field region to retain the target biological material through direct current or alternating current generation.

53. The device of any one of claims 46-52, further comprising a well in the DEP low-field region to retain the target biological material during washing or flushing steps.

54. Isolated biological material that is selectively retained on a DEP low-field region of an alternating current electrokinetic device.

55. Use of the isolated biological material of claim 54 for detecting the presence of one or more biomarkers in a sample from which the isolated biological material has been obtained.

56. A system for isolating target biological material from a sample, the system comprising:
a. a device comprising a plurality of alternating current (AC) electrodes within the housing, the AC electrodes configured o be selectively energized to establish dielectrophoretic (DEP) high-field and dielectrophoretic (DEP) low-field regions, whereby AC electrokinetic effects provide for separation of the target biological material from other entities in the sample at the DEP low-field region of the device;
wherein the biological material is at least 800 nm in diameter.
